(19) United States
(12) Patent Application Publication
(10) Pub. No.: US 2026/0341464 A1
(43) Pub. Date: Oct. 29, 2026

(54) System and Method for Generating Personalized Metabolic Health Plans with Adaptive Avatar and Real-Time Recalibration

(71) Applicants: Sky Christopherson, Tucson, AZ (US); Clyde Wilson, Tucson, AZ (US); David Christopherson, Tucson, AZ (US)

(72) Inventors: Sky Christopherson, Tucson, AZ (US); Clyde Wilson, Tucson, AZ (US); David Christopherson, Tucson, AZ (US)

(21) Appl. No.: 19/380,939

(22) Filed: Nov. 5, 2025

Related U.S. Application Data
(63) Continuation-in-part of application No. 19/189,030, filed on Apr. 24, 2025, now Pat. No. 12,518,865.
(63) Continuation-in-part of application No. 19/189,025, filed on Apr. 24, 2025, now Pat. No. 12,573,507.

(51) Int. Cl. G16H 20/60; G06N 3/004

(52) U.S. Cl. CPC G16H 20/60; G06N 3/004

(57) ABSTRACT

Systems and methods are disclosed for generating a personalized metabolic health plan for a user by receiving input data comprising user age, sex, body mass, goals, workout intensity, non-exercise activity thermogenesis, sleep timing and quality, stress response, and dietary patterns; estimating resting metabolic rate using a weighted combination of metabolic rate estimation based on user physical parameters and organ system weight-based models; determining a 24-hour calorie utilization profile by mapping metabolic rate slope and correlating feeding and activity events; quantifying nutrient and metabolic gaps, including caloric deficit, macronutrient flow, hydration, and nourishment bottlenecks; adjusting recommended targets for longevity, health, weight (fat) loss, lean tissue gain, and fitness by applying goal-specific tunable coefficients; calculating a probability-ranked list of lifestyle interventions using additive, multiplicative, and weighted coefficient-based mathematical operations; generating a visually accessible metabolic plan including at least one of: three to six key daily actions, pie charts of food or activity groups, and detailed units in grams, sets, or time intervals; and providing an adaptive avatar interface that engages the user in a conversational format, simplifies outputs, monitors adherence, and recalibrates recommendations in real time based on updated user data.

This application is a continuation-in-part of U.S. application Ser. Nos. 19/189,025 and 19/189,030, the contents of which are incorporated by reference.

### BACKGROUND OF THE INVENTION

Chronic metabolic disorders such as obesity, type 2 diabetes, and metabolic syndrome pose escalating public health challenges linked to sedentary behavior, irregular eating patterns, and circadian disruption. Recent advances in wearable sensors, continuous glucose and metabolic monitoring, and mobile health platforms have enabled richer, longitudinal capture of physiological, behavioral, and contextual data, supporting more individualized risk assessment and intervention. Complementary progress in computational modeling and machine learning has enhanced prediction of glycemic responses, energy balance, and sleep-wake dynamics over daily cycles, while digital coaching paradigms—including embodied avatars and conversational agents—have been deployed to motivate adherence and support behavior change. Despite these developments, variability in individual responses and the complexity of integrating multimodal, time-resolved data into actionable, user-friendly guidance remain central challenges driving ongoing research and product innovation in personalized metabolic health management.

### SUMMARY OF THE INVENTION

A computer-implemented method generates a personalized metabolic health plan for a user comprises receiving input data comprising user age, sex, body mass, goals, workout intensity, non-exercise activity thermogenesis, sleep timing and quality, stress response, and dietary patterns. In one aspect, the method estimates resting metabolic rate using a weighted combination of metabolic rate estimation based on user physical parameters and organ system weight-based models.

In one aspect, the method determines a twenty-four hour calorie utilization profile by mapping metabolic rate slope and correlating feeding and activity events. In one aspect, the method quantifies nutrient and metabolic gaps, including caloric deficit, macronutrient flow, hydration, and nourishment bottlenecks. In one aspect, the method adjusts recommended targets for longevity, health, weight (fat) loss, lean tissue gain, and fitness by applying goal-specific tunable coefficients. In one aspect, the method calculates a probability-ranked list of lifestyle interventions using additive, multiplicative, and weighted coefficient-based mathematical operations. In one aspect, the method generates a visually accessible metabolic plan including at least one of three to six key daily actions, pie charts of food or activity groups, and detailed units in grams, sets, or time intervals. In one aspect, the method provides an adaptive avatar interface that engages the user in a conversational format, simplifies outputs, monitors adherence, and recalibrates recommendations in real time based on updated user data.

In another aspect, a computer-implemented method generates a personalized metabolic health plan by receiving user-specific inputs (age, sex, body mass, goals, workout intensity, non-exercise activity thermogenesis, sleep timing and quality, stress response, and dietary patterns), estimating resting metabolic rate using a weighted combination of physical-parameter equations and organ-system weight-based models with dynamic equation selection based on BMI and activity, and deriving a 24-hour calorie utilization profile by mapping metabolic-rate slope to time-stamped feeding and activity events.

The method quantifies nutrient and metabolic gaps—including caloric deficit, macronutrient flow, hydration, and nourishment bottlenecks such as protein timing, carbohydrate distribution, omega fatty acid ratios, and vegetable intake—and divides daily macronutrient targets by meal frequency to produce per-meal or per-hour targets, adjusted by goal-specific tunable coefficients for longevity, health, fat loss, lean tissue gain, and fitness. A probability-ranked list of lifestyle interventions is calculated using additive, multiplicative, and weighted coefficient operations that score positive actions and penalize bottlenecks; the system generates visually accessible plans with three to six key daily actions, charts, and detailed units (grams, sets, time), provides portion guidance via hand metrics, tailors exercise refueling by body weight and absorption windows, ranks top adjustments, weights future priorities by user feedback, and presents an adaptive avatar interface (graphical, voice, or text) that engages the user, monitors adherence, and recalibrates recommendations in real time based on updated data and detected deviations while offering selectable lifestyle scaffolds such as Mediterranean or extreme carbohydrate variation.

In a further aspect, a method for an avatar to determine a personalized metabolic plan for a user includes:
receiving user data, the data comprising age, sex, body weight, body height, activity level, body mass index, metabolic goals, sleep timing, sleep quality, stress patterns, and dietary intake patterns;
estimating a resting metabolic rate for the user by selectively applying one or more validated metabolic equations, each equation configurable to utilize user-specific physical inputs and optionally forming a weighted average of outputs; wherein the equations comprise at least weight, height, age, and sex variables;
calculating an estimated total daily metabolic rate by incorporating the resting metabolic rate and adjusting for user-specific activity levels, non-exercise activity thermogenesis, and weighted goal preferences;
determining calorie utilization for a defined 24-hour period, by dividing the total daily metabolic rate by 24 to produce an hourly metabolic rate, and mapping user feeding events and activity events on a timeline, wherein feeding events are assigned upward graphical indicators sized proportional to caloric intake, and activity events are assigned downward graphical indicators sized proportional to caloric expenditure;
quantifying caloric gaps by comparing daily caloric intake at feeding and snacking events with total expenditure, such that cumulative deficits or surpluses are represented graphically over the 24-hour period from postprandial sleep state through daily activity cycles;
repeating the gap quantification and mapping process for individual macronutrients including protein and carbohydrate, by calculating user baseline needs for each macronutrient, computing a healing or recovery adjustment as a function of recent injury, physical stress or exercise, and determining timing and distribution across meals, snacks, and feeding events;
estimating meal and snack portion sizes for caloric and macronutrient targets using visual and physical measures including thumb, palm, and first volumes, and aligning portion guidelines with user preferences and patterns;
assigning tunable goal values for each metabolic objective, the objective comprising at least weight and fat loss, longevity, health, fitness, healthy weight gain, and lean tissue protection, each goal associated with a user-adjustable Z-value indicative of relative importance; wherein Z-values are used quantitatively to increase or decrease recommended caloric and nutrient targets by fixed or proportionate amounts;
calculating nutrient bottlenecks and gaps including protein, carbohydrate, hydration, vegetable intake, and omega fatty acid intake; for each bottleneck, determining a recommended intake range and identifying food sources and portion sizes to address the gap;
estimating hydration requirements in relation to metabolic rate, recommending a baseline target of liquid intake per 1000 calories of estimated metabolic rate, and generating timing recommendations for intake distribution throughout the day;
mapping timing for slow-digesting and fast-digesting foods based on digestion rates for protein, carbohydrate, and fat sources, and determining minimum and ideal hourly nutritional requirements by dividing daily targets by estimated effective provision periods between feedings;
calculating and visualizing vegetable and fatty acid goals, including baseline and Z-value-driven increases, and presenting results as portion guidelines (in cups, palms, tablespoons);
recommending and displaying individualized metabolic meal plans for the user, including selection and arrangement of food libraries for each meal and snack event, with checklists and graphical meal composition options;
scoring user nutritional and metabolic adherence using carrot versus stick scoring algorithms, wherein positive actions are counted additively and major bottlenecks result in multiplicative penalties, producing a composite metabolic health score;
recommending movement and exercise interventions, including activity break scheduling, specific exercise and activity recommendations are modulated by user-selected Z-values corresponding to their metabolic goals;
recommending sleep, stress, eating, hydration, movement, and psychological patterns for the user, prompting user enrollment of individual priority factors for each category, ranking factors by combined importance and difficulty, and outlining top-priority actions with recommended solutions for common barriers;
generating an adaptive, visually accessible output comprising at least: (i) daily key accomplishment checklists; (ii) graphical pie charts illustrating proportional metabolic targets; and (iii) detailed units of food portions or activity sets in grams, ounces, thumb/palm/fist equivalents, minutes, or repetitions; and
providing real-time tracking and adaptive recalibration of recommendations and targets, updating outputs and priority interventions as user data, adherence, or underlying conditions (including sleep quality and activity deviation) change.

Advantages of one implementation may include one or more of the following:
1. Improved personalization: combines demographic, anthropometric, behavioral, physiological, and contextual inputs to generate individualized metabolic plans that account for inter-subject variability.
2. Enhanced resting metabolic rate estimation: uses a weighted combination of conventional physical-parameter estimators and organ-weight based models to improve accuracy over single-method approaches.
3. Twenty-four hour temporal fidelity: maps metabolic rate slopes and correlates feeding and activity events to produce a time-resolved calorie utilization profile that captures circadian and activity-driven fluctuations in energy expenditure.
4. Multimodal gap detection: quantitatively identifies caloric deficits, macronutrient flow imbalances, hydration shortfalls, and nourishment bottlenecks to highlight specific, addressable metabolic vulnerabilities.
5. Goal-specific tuning: applies adjustable coefficients for longevity, health, fat loss, lean tissue gain, and fitness goals to tailor targets and tradeoffs according to individual preferences and priorities.
6. Prioritized interventions: generates a probability-ranked list of lifestyle interventions using additive, multiplicative, and weighted coefficient operations to recommend actions most likely to achieve user goals.
7. Actionable outputs: presents recommendations in simple, implementable units (grams, sets, minutes, time windows) and compact daily action lists (three to six key items) to facilitate user adherence.
8. Clear visualizations: produces pie charts and other visually accessible summaries of food and activity groups to improve user understanding and decision making.
9. Adaptive conversational interface: employs an avatar or conversational agent to simplify complex outputs, provide coaching, and engage users in naturalistic dialogue.
10. Real-time recalibration: continuously monitors adherence and updated sensor or user-reported data to dynamically adjust recommendations and maintain alignment with evolving user state.
11. Improved engagement and adherence: combines prioritized, time-sensitive recommendations with an interactive avatar to increase salience and likelihood of behavior change.
12. Interoperability with wearables and platforms: ingests continuous glucose, activity, sleep, and other sensor streams to leverage existing consumer and clinical devices.
13. Explainability and transparency: uses modular, coefficient-based operations and explicit scoring to provide interpretable rationales for recommended interventions.
14. Scalability and extensibility: modular computational design supports integration of new sensors, models, or therapeutic goals without fundamental redesign.
15. Support for clinical and population use: provides individualized plans suitable for direct consumer use and structured outputs that can inform clinicians, coaches, or research studies.
16. Efficient decision making: reduces cognitive load by distilling complex, multimodal data into a small set of high-impact, ranked actions.
17. Accommodation of circadian and behavioral patterns: adjusts timing and composition recommendations to align with sleep-wake cycles, meal timing, and daily activity patterns.
18. Potential for improved metabolic outcomes: by aligning interventions with individualized risk factors, temporal metabolism, and adherence likelihood, the system may increase the effectiveness of lifestyle modification for weight, glycemic control, and overall metabolic health.
19. Privacy-preserving deployment options: compatible with locally hosted or encrypted, consented cloud architectures to protect sensitive health data.
20. Continuous learning and population refinement: framework permits model updates from aggregated, de-identified user data to refine predictions and intervention rankings over time.

### BRIEF DESCRIPTION OF DRAWINGS

FIG. 1 illustrates a flowchart for generating a personalized metabolic health plan, from receiving user data to adaptive avatar-driven real-time recalibration.

FIG. 2 depicts a hand holding a smartphone displaying an avatar on metabolic guidance to help a user after exercise.

FIG. 3A-3F shows an exemplary avatar based coaching system.

### DETAILED DESCRIPTION OF THE INVENTION

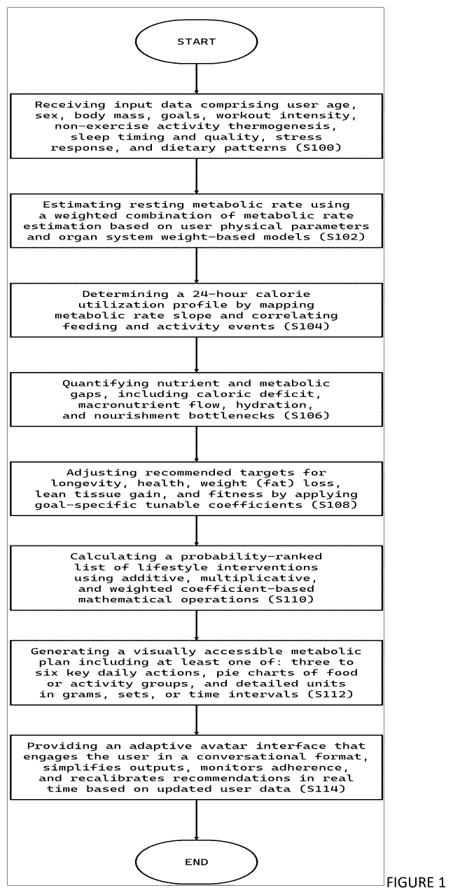

FIG. 1 illustrates a flowchart for generating a personalized metabolic health plan, from receiving user data to adaptive avatar-driven real-time recalibration. The method generates a personalized metabolic health plan by receiving user-specific input data, estimating basal and dynamic energy requirements, mapping twenty-four hour calorie utilization, quantifying nutrient and metabolic gaps, adjusting targets according to user goals, ranking lifestyle interventions by probability, and presenting an adaptive, conversational avatar interface that monitors adherence and recalibrates recommendations in real time. The method integrates user age, sex, body mass, goals, workout intensity, non-exercise activity thermogenesis, sleep timing and quality, stress response, and dietary patterns to produce actionable daily guidance including prioritized actions, graphical summaries, and precise units for food, activity, and hydration.

User interface considerations include reducing friction and supporting progressive disclosure of information. The avatar's primary view emphasizes prioritized actions. Secondary screens allow a user to view full timelines, macronutrient tables, and historical progress graphs. Notifications and reminders are configurable and can be scheduled to coincide with predicted windows of optimal impact (for example, prompting protein intake within an hour after resistance exercise). The avatar is further configured to provide motivational dialogue, enable micro-goal setting, and deliver contextual tips based on user routines.

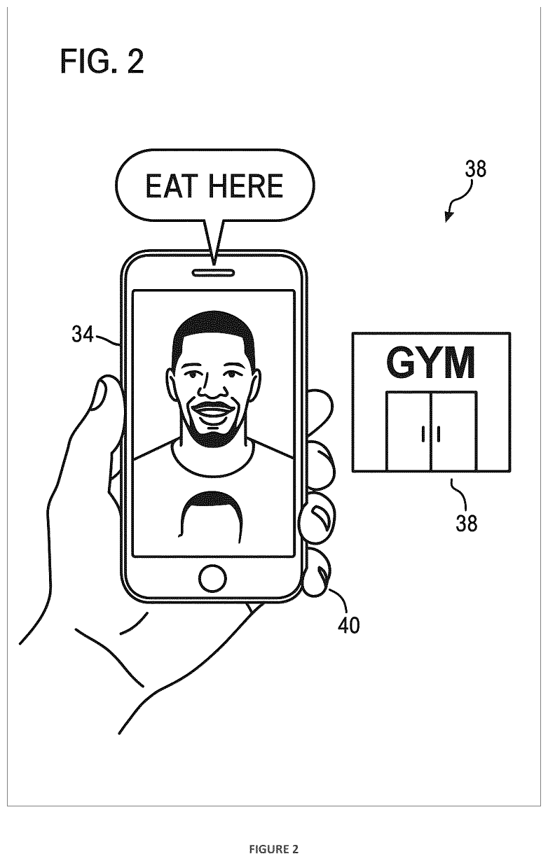

The system supports extensibility through plugin components for specialized analytics, including continuous glucose response modeling, rehabilitation program administration, and integration with clinical workflows. In clinical or research embodiments, de-identified, aggregated data are collected and used to refine population-level parameters, adjust tunable coefficients, and enhance organ-weight apportionment models. The system also incorporates configurable safety rules and provides clinician override where appropriate, for example to prevent recommendations that would violate medically indicated energy intake limits. The information is delivered using an avatar as shown in FIG. 2, which depicts a smartphone displaying an avatar providing metabolic guidance to help a user after exercise. In the example of FIG. 2, device 34 renders the avatar with an audiovisual message based on the flow of FIG. 1 and conveying an actionable instruction and serves as the client through which plan updates, adherence monitoring, and real-time recalibration are delivered.

The process of FIG. 1. is a comprehensive and adaptive framework for estimating and managing human metabolic flow, nutrition, exercise, sleep, and stress, with the primary objective of guiding individuals toward optimal health outcomes based on validated scientific equations and scalable lifestyle interventions. Central to the algorithm is the idea that metabolic rate estimation should not rest on static calorie targets but rather on dynamic, context-sensitive factors such as subjective food portioning (using thumb, palm, and first units), assessment of meal sizes, inherent protein within calorie counts, and personalized hydration needs. Validated metabolic equations, most notably the widespread use of the Mifflin-St. Jeor formula for calculating resting metabolic rate (RMR), which incorporates variables such as weight, height, age, and a sex-based modifier. Simplicity of use is achieved through nonlinear approximations for average body weights, while the most accurate estimations are derived from organ system weight calculations, with a weighted average adjusting to reflect individual goals relating to fat loss, longevity, health, fitness, or healthy weight gain. The user's specific goal is translated into a quantitative “Z-value,” where shifts in caloric intake targets (UP or DOWN) are directly computed as a function of these Z-values, allowing caloric prescriptions to be personalized and optimally tuned for body composition changes and metabolic health.

The framework charts caloric gaps and then extends these calculations to discrete nutrient gaps, thus creating a broader “metabolic gap” that forms the basis for long-term health monitoring and intervention. Metabolic flow is mapped visually throughout the day in a sequence beginning with the post-sleep, fully fed state and progressing through patterns of feeding and exercise, where upward arrows represent meals and downward arrows indicate energy expenditures from physical activity. The document provides a detailed breakdown of how energy percentage is apportioned during varying levels of exercise intensity—low, medium, high, and very high—defined by the duration an individual could sustain such effort before exhaustion, and sized as percentages of the total daily metabolic rate. By dividing the total daily energy expenditure by twenty-four, users can approximate their hourly metabolic rate, which serves as a baseline for hourly nutrient requirements. This approach is then repeated for proteins and carbohydrates, with a focus on the timing and flow necessary to protect lean tissue and maintain stable blood sugar and amino acid availability. Protein requirements are context-sensitive and tunable (0.4-0.8 grams per kilogram of body weight for baseline, up to 1.2 grams per kilogram for healing), with adjustments accounting for recovery from injury, pregnancy, lactation, and high levels of exercise. The digestion rates of common protein foods are listed to inform optimal timing and distribution across meals.

Carbohydrate needs are linked to brain function, physical healing, and movement, with minimum and ideal daily targets calculated and broken into hourly requirements to facilitate even macronutrient delivery. There is significant attention paid to the types of foods that deliver carbohydrates, their botanical and functional definitions, digestion rates, and the recommended portion sizes for leafy vegetables, fruits, legumes, and starchy foods. Hydration guidance is derived from metabolic rate, with targets set at 32 ounces or 1 liter per 1,000 calories consumed, and the algorithm encourages an even flow of water intake throughout the day, especially between meals and upon waking.

Nourishment, as described, involves the integration of vegetables and omega fatty acids for a fully encompassing metabolic nutrition blueprint. The document references dietary guidelines and recommends scalable intake targets for omega-3, omega-6, and omega-9 fats, with individual goal-based Z values driving percentage increases in portions above standard recommendations. Visual delivery options for meal planning range from simple checklists (three key accomplishments per day), to pie charts indicating relative food group proportions, to highly detailed tables providing units of grams, ounces, or hand-based measurements. The algorithm provides further granularity for tracking and adjusting metabolic priorities, such as time between meals, food types for snacks, and post-exercise refueling strategies. For physical activity, movement guidelines are structured according to the same personalized Z-value hierarchy, specifying amounts and intensities of exercise-active sitting intervals, movement breaks, brisk cardio, strength, power, and endurance training-again tuned for the individual's primary health, weight, and fitness objectives.

Stress and sleep management are tightly linked within the system to eating, hydration, movement, and psychological patterns, each presented as potentially critical contributors to sleep quality and overall metabolic wellness. The user is prompted to identify factors most relevant to their own well-being and prioritize these for actionable change, with the algorithm supporting the selection and ranking of six key factors in order of maintenance difficulty and personal importance. This results in a metabolic priority program emphasizing the most achievable and highest-impact changes.

In one implementation, an additive (“carrot”) and multiplicative (“stick”) scoring systems to evaluate user performance, with coefficients that reflect the interconnectedness of nutrition, movement, sleep, and stress levers. It includes provisions for individualized adjustment and decision-support, such as default modifications for popular diets (e.g., Mediterranean diet) and adaptation for groups or social “Fox Den” team challenges. The framework is designed for digital and app-based delivery, with users empowered to scaffold programs according to their chosen lifestyle patterns, skill levels, and health needs, ensuring that even extreme dietary or activity patterns can be optimized safely through scientific personalization and adaptive recommendations.

Turning now to FIG. 1, at S100 the process receives input data comprising user age, sex, body mass, goals, workout intensity, non-exercise activity thermogenesis, sleep timing and quality, stress response, and dietary patterns. In the embodiment corresponding to S100, the system receives the input data via one or more interfaces, including but not limited to user-entered responses through a graphical user interface, sensor-derived measurements obtained from wearable devices, and externally provided data accessed through application programming interfaces or import routines, and the received data are associated with a user profile and time-stamped upon receipt. The data received at S100 are validated and normalized as needed to conform to stored data formats and units, and are stored in volatile or non-volatile memory for subsequent processing steps; missing, inconsistent, or out-of-range values are flagged for correction or supplemented by inferred estimates according to predefined rules. The receiving operation at S100 is configured to accept initial baseline data and to accept periodic or event-driven updates to the same data elements so that downstream computations use the most recent validated input.

The estimation of resting metabolic rate is performed by combining a conventional metabolic rate estimate based on the user's physical parameters with organ system weight-based models to yield a weighted resting metabolic rate, as recited in step S102. In S102, the method applies tunable weighting coefficients to fuse an empirical formula driven by age, sex, height, and body mass with organ-specific metabolic contributions scaled to organ weights, thereby producing a calibrated baseline metabolic rate. The output of S102 serves as the reference resting metabolic rate that informs subsequent operations such as determining a twenty-four hour calorie utilization profile (S104) and quantifying nutrient and metabolic gaps (S106).

The method determines a twenty-four hour calorie utilization profile by mapping metabolic rate slope and correlating feeding and activity events (reference label S104). In S104, temporal changes in metabolic rate are modeled across a full diurnal cycle by computing the slope of estimated metabolic rate over time and aligning recorded or projected feeding times and physical activity events with those temporal changes; the resultant profile quantifies periods of increased and decreased energy expenditure, supports identification of the timing and magnitude of calorie utilization, and enables upstream tailoring of nutrient timing and activity prescriptions to match individual metabolic dynamics.

Step S106 quantifies nutrient and metabolic gaps by calculating discrepancies between the twenty-four hour calorie utilization profile and recorded or reported intake to identify caloric deficit magnitudes and timing. S106 analyzes macronutrient flow by comparing actual macronutrient intake rates against target distributions and by mapping temporal alignment of macronutrient availability with periods of elevated metabolic demand, thereby identifying deficits or surpluses in protein, carbohydrate, and fat provision. S106 assesses hydration status against individualized hydration requirements derived from body mass, activity intensity, and environmental factors, and identifies nourishment bottlenecks by detecting recurrent periods within the daily profile where nutrient delivery is insufficient to meet metabolic or recovery needs. S106 receives inputs from the initial data collection and the twenty-four hour utilization profile, outputs quantitative gap metrics in standard units (for example calories, grams, liters, and time windows), and provides these metrics to downstream operations that adjust targets (S108), rank interventions (S110), and generate user-facing plans (S112).

Step S108 adjusts recommended targets for longevity, health, weight (fat) loss, lean tissue gain, and fitness by applying goal-specific tunable coefficients. In S108, baseline targets derived from metabolic estimates and gap analyses are scaled by a set of tunable coefficients that encode the user's prioritized objectives and clinically informed trade-offs; each coefficient is applied to caloric, macronutrient, and activity targets to bias recommendations toward longevity, general health optimization, fat reduction, lean mass accretion, or performance improvements, and the coefficients are configurable to refine the balance among competing goals.

Based on the adjusted targets, the method calculates a probability-ranked list of lifestyle interventions using additive, multiplicative, and weighted coefficient-based mathematical operations to rank interventions by expected benefit and feasibility (S110). The system generates a visually accessible metabolic plan that presents three to six key daily actions, graphical representations such as pie charts of food or activity groups, and detailed quantitative targets expressed in grams, sets, or time intervals to support implementation and tracking (S112). An adaptive avatar interface engages the user in a conversational format, simplifies outputs for comprehension, monitors adherence, and recalibrates recommendations in real time using updated user data to maintain alignment with evolving status and goals (S114).

The step identified by reference label S110, calculating a probability-ranked list of lifestyle interventions, computes a score for each candidate intervention by combining feature contributions with additive weighted terms, incorporating multiplicative interaction factors where interactions between features affect outcome, and applying coefficient-based scaling to reflect goal-specific priorities. The computed scores are normalized to form a probability distribution over interventions, and the interventions are ordered according to their resulting probabilities to produce a ranked list; associated probability values and contributing factors are made available for downstream presentation and selection.

At step S112, the method generates a visually accessible metabolic plan that presents at least one of three to six key daily actions, pie charts of food or activity groups, and detailed units in grams, sets, or time intervals. The key daily actions are concise, prioritized behaviors derived from the user's metabolic profile and goals and are presented with explicit numeric targets and scheduling guidance to facilitate adherence. The pie charts convey aggregate distributions of foods or activities to illustrate macronutrient or activity-group composition over a selected interval. The detailed units provide precise, actionable measurements-such as gram-based nutrient targets, set and repetition counts for resistance training, and timed intervals for meals or activities-so that recommended interventions can be implemented and tracked with specificity.

Reference label S114 corresponds to providing an adaptive avatar interface that engages the user in a conversational format, simplifies outputs, monitors adherence, and recalibrates recommendations in real time based on updated user data. The adaptive avatar presents prioritized actions and visual summaries, receives user responses and adherence indicators, and responsively updates the personalized metabolic plan to reflect incoming user data and measured outcomes.

Intervention priority adjustments are computed by reweighting the intervention scoring model to favor restorative and reduced-stress interventions while demoting interventions that impose substantial metabolic or neuromuscular load. Practically, the multiplicative modulators described for S110 are updated: an intervention stress factor S_stress is applied so that interventions producing acute physiological stress receive a downscaling multiplier m<1, whereas restorative actions with minimal stress receive an upscaling multiplier m>1. For example, following a significant sleep timing or quality deviation, recommended intensity for planned resistance sessions is scaled down (e.g., target relative intensity reduced by 10-30%), volume is reduced (sets or repetitions decreased by a defined percentage), and recommendations for protein timing, hydration, and gentle activity or nap opportunities are elevated in priority. The recalibration routine also re-executes the 24-hour calorie utilization mapping (S104) with updated activity intensity expectations and altered postprandial dynamics that reflect the shifted recovery window, producing revised nutrient gap quantifications (S106) and adjusted targets (S108) as necessary.

Recalibration can be immediate or staged depending on severity and persistence. For a single-night mild deviation, the system can apply a transient recalibration that decays back to baseline within a predefined number of cycles (for example, 24-72 hours) unless deviations persist. For repeated or chronic deviations, the system is configured to update persistent coefficient values and adjust circadian-aligned target schedules. The system records each recalibration event with timestamps, the computed S_sleep, the actions taken (recovery window adjustment, intervention reprioritization), and the rationale to support transparency and enable user review.

Structured resistance training is prescribed with parameters including frequency (sessions/week), intensity (% of one-repetition maximum or % of maximal voluntary contraction), volume (sets×repetitions), and rest intervals. For example, to preserve lean mass while achieving an average daily deficit of X kcal, the system recommends resistance training 3 sessions/week at 70-85% 1RM, 3-5 sets per major movement, and 6-12 repetitions per set, with progressive overload rules encoded as weekly volume or intensity increments.

Aerobic recommendations are expressed in heart-rate or metabolic equivalent (MET) zones and duration. For example, the system prescribes moderate-intensity aerobic activity at 50-70% of heart rate reserve for 30-60 minutes per session, 3-5 sessions per week, while vigorous-intensity interval training (VIIT) is prescribed as 2 sessions/week of intervals at 85-95% HRmax for 10-20 minutes total work time.

If the user's primary goal is lean tissue gain, the system applies coefficients that favor a positive energy balance targeted to muscle synthesis while minimizing excessive adiposity. Exercise prescriptions emphasize higher resistance training frequency (e.g., 3-6 sessions/week), progressive volume increases, and intensity prescriptions such as multiple weekly sessions per muscle group at 65-85% 1RM with target weekly sets per muscle group calibrated to the user's training age. The system couples these resistance prescriptions with timing and macronutrient flow recommendations to maximize muscle protein synthesis, specifying pre- and post-exercise protein amounts in grams and timing windows (e.g., 20-40 g protein within 0-2 hours post-session) and carbohydrate targets for glycogen replenishment.

If the user's primary goal is longevity or general health, the system applies coefficients prioritizing moderate-intensity aerobic volume, resistance exercise for preservation of function, balance and mobility work, and avoidance of excessive training stress. Prescriptions specify at least 150 minutes per week of moderate-intensity aerobic activity or 75 minutes per week of vigorous activity, distributed across the week in sessions aligned with circadian metabolic patterns. Timing rules consider sleep timing and quality (from S100) and the metabolic slope S(t) (from S104) so that higher-intensity sessions are scheduled at times of optimal metabolic responsiveness, while accounting for user schedule constraints and recovery needs.

Timing of exercise is determined by aligning the 24-hour calorie utilization profile U(t) with feeding events and circadian factors. The system computes a temporal score for each potential exercise time t based on U(t), user-reported sleep timing and quality, and anticipated postprandial windows. For example, strength training is scheduled in time windows where nutrient availability and energy are favorable for performance and recovery (e.g., 1-3 hours after a protein-containing meal), whereas light-intensity NEAT sessions are suggested during early-morning windows when U(t) indicates a substrate utilization profile favorable for fat oxidation. The temporal score is combined with user preferences and constraints through weighted scoring; the system then ranks candidate time slots and selects an optimized schedule that maximizes the aggregate score while respecting maximum allowable daily and weekly loads.

Intensity modulation uses user-specific physiological proxies when available. If device sensors or user inputs provide heart rate, VO2max, or perceived exertion data, intensity targets are framed in heart rate reserve %, VO2%, or RPE ranges. Where direct physiological measures are absent, intensity is estimated from user fitness level, age, and activity history using conservative scaling factors. The system translates intensity into expected metabolic rate multipliers and uses these to predict ΔE_k(t). Intensity is also tuned to the user's stress response and recovery status: elevated stress biomarkers or poor sleep quality reduce recommended intensity and increase recovery time; conversely, good recovery indicators permit higher intensities or volume.

Duration assignment is calculated to achieve a prescribed caloric or stimulus target at the selected intensity. For caloric objectives, duration D_k=required_calories/estimated_energy_rate(I_k, user), wherein estimated_energy_rate incorporates a MET-equivalent conversion adjusted for the user's mass and RMR_est. For stimulus-based objectives (e.g., hypertrophy volume), duration is computed from sets, repetitions, and inter-set rest durations aggregated across prescribed movements. Minimum effective dose constraints and maximum allowable load constraints are enforced to reduce injury risk; for example, a minimum resistance session duration is defined as two compound movements totaling at least 15 minutes of dedicated strength stimulation, and maximum session duration is capped based on user recovery metrics.

The system incorporates personalization layers that tailor exercise content based on user experience, comorbidities, and available equipment. A novice is provided with simplified movement selections, progressive onboarding, reduced initial intensity, and an increased frequency of gentle NEAT prompts, while an experienced user receives complex periodized programs tailored to advanced objectives. Equipment availability is addressed by offering alternative exercises that achieve the same biomechanical targets. Safety checks assess user age, medical conditions, recent injuries, and medications to exclude or modify recommendations that carry increased risk and to prompt medical clearance when indicated.

Continuously updates exercise prescriptions based on adherence and sensor-derived feedback. When a user completes a session, recorded metrics (duration, heart rate response, perceived exertion) are compared to expected values and used to update models for energy expenditure and fitness progression. Non-adherence is addressed by the avatar interface via conversational prompts that solicit reason codes (time constraints, fatigue, pain) and propose alternative interventions with comparable P_k but reduced time or intensity burden. The system recalculates target distributions and can reassign energy expenditure to NEAT or dietary adjustments when structured exercise adherence falls below threshold levels.

Examples of adaptive behavior include automatic down-regulation of exercise intensity when consecutive sessions show elevated resting heart rate or poor sleep, and conversely the introduction of progressive overload when performance metrics exceed predicted thresholds. Macronutrient timing is coordinated with resistance sessions to enhance recovery and hypertrophy, specifying gram-level protein targets per session and carbohydrate dosing before and after vigorous aerobic work to support glycogen repletion.

Example: For a 70 kg user (W=70) completing a 60-minute vigorous interval session with substantial glycogen depletion and planning a second session 6 hours later, the system might compute Carb_rec_window=70*0.9*1.5*1.0≈94.5 g carbohydrate to be consumed over the first 2-4 hours post-exercise, implemented as three 30-35 g boluses at 0-30 minutes, 60-90 minutes, and 150-180 minutes post-exercise. Protein recommendation Prot_rec_meal=70*0.3≈21 g within 45 minutes post-exercise, followed by subsequent 20-30 g protein feedings every 3-4 hours. Hydration recommendation Hydr_rec=1.0 L fluid replacement*1.25≈1.25 L in the first 4 hours with electrolyte inclusion after prolonged sweating. These numeric suggestions are provided with recommended ranges and are adjusted by the goal coefficients from S108 (e.g., carbohydrate amounts are increased if performance for the next session is prioritized, and reduced if fat loss is prioritized and the next session is not imminent).

Integration of the exercise-refueling recommendations into the overall plan ensures that the refueling energy and macronutrients are included in the 24-hour calorie utilization profile CE(t) and in the nutrient gap analyses. The probability-ranked intervention list (S110) will consider the refueling intervention's feasibility and expected effect size; if adherence probability is insufficient, alternative intervention combinations (e.g., pre-sleep protein, altered training scheduling to allow longer recovery) will be presented. The avatar (S114) presents the refueling recommendation as a concise, minimal-effort action card (e.g., “Within 45 minutes: 21 g protein+30 g carbohydrate; drink approximately 400 mL electrolyte beverage”) and requests confirmation or image capture. Upon confirmation, downstream modules update nutrient flow estimates and decrease the severity of the flagged refueling bottleneck.

MR(t)=RMR_combined*f_circadian(t)+MR_activity(t)+MR_postprandial(t),

Where f_circadian (t) is a circadian modulation function (for example, a cosine or spline-based function parameterized by sleep timing and chronotype), MR_activity(t) is activity-induced expenditure derived from activity intensity and duration, and MR_postprandial(t) is the thermic effect of feeding assigned to intervals following reported feeding events. The metabolic-rate slope across intervals is computed by finite differencing MR(t) and used to temporally align substantial feeding or activity events with metabolic increases and decreases, enabling computation of anticipated energy expenditure per interval and aggregated daily energy utilization curves.

User feedback on perceived ease versus perceived impact of metabolic actions is collected and used to weight future recommendation priority and difficulty in subsequent plans. For each recommended action a that is presented to the user, the system prompts for feedback along at least two dimensions: perceived ease E_a (for example on a Likert scale 1-5, where 1 indicates very difficult and 5 indicates very easy) and perceived impact I_a (for example on a Likert scale 1-5, where 1 indicates negligible impact and 5 indicates major impact). The system also captures adherence outcomes (binary or graded adherence metrics) and objective outcome measures where available (for example weight change, measured strength gains, or biomarker changes) to corroborate subjective feedback.

Subjective feedback is normalized to [0,1] as e_a=(E_a−1)/4 and i_a=(I_a−1)/4. A feedback-derived weighting factor W_a is computed to reflect user preference and experienced utility. One example formulation is W_a=α·i_a+(1−α)·e_a, where α∈[0,1] is a personalization parameter that determines the relative importance of perceived impact versus ease for that user. The parameter a can be initialized from a user-stated preference (for example, a user indicates a bias toward actions with greater impact despite increased difficulty) and updated over time based on observed adherence and outcome correlations. Alternatively, a multiplicative formulation W_a=i_a\acute{ }γ·e_a\ddot{ }(1−γ) can be used to penalize actions with reduced ease even when impact is rated greater, where γ controls the curvature of preference.

Feedback is integrated into the intervention scoring pipeline by modifying the difficulty penalty and the intervention utility. Specifically, the difficulty parameter D_i in the utility formulation is adjusted to reflect perceived ease: D_i_new=D_i*(1−μ*ē_i) where ē_i is an exponential moving average of reported ease for intervention i and μ∈[0,1] is a sensitivity coefficient that governs how rapidly perceived ease reduces the effective difficulty. The expected effectiveness E_i is updated to incorporate perceived impact via E_i_new=E_i*(1+v*(l̅_i−i_ref)), where l̅_i is a moving average of reported impact for intervention i, i_ref is a reference impact (for example population average), and v is a learning rate. The composite utility U_i is recalculated using the updated D_i_new and E_i_new, and rankings are adjusted accordingly for subsequent plans.

Feedback-driven personalization operates across multiple timescales. Immediate updates use incoming feedback to re-rank interventions for the next plan-generation cycle. Medium-term personalization aggregates feedback over days to weeks and updates the personalization parameter a and the moving averages ē_i and l̅_i using exponential smoothing (for example ē_i←βē_i+(1−β) e_a, with β close to 1 for slow adaptation). Extended-horizon learning employs Bayesian updating or reinforcement-learning-style value-function updates that incorporate objective outcome measures to infer true intervention efficacy and to disambiguate placebo-like perceived impact from objective effect. When perceived impact and objective outcomes diverge, the system down-weights purely subjective signals in favor of objective measures while continuing to honor user preference when adherence is strongly correlated with perceived ease.

Perceived difficulty is also used to calibrate future recommended action difficulty tiers. The system maintains difficulty tiers (for example, easy, medium, challenging) for each action or action family. Thresholds for tier assignment are adaptively shifted based on aggregated user ease feedback and observed adherence. For example, if an action consistently receives reduced ease scores (ē_i below a preselected threshold), that action is moved to a more demanding tier for that user and substitutes of equivalent expected impact but easier to perform are preferentially selected. Conversely, actions that users rate as easy and impactful are promoted and more likely to be presented as priority recommendations.

Monitoring adherence and recalibrating recommendations are continuous processes. Adherence metrics are fused from self-report, passive sensor signals (for example steps, heart rate, active minutes), and contextual information (for example calendar events). When adherence falls below a goal-specific threshold, the system analyzes recent feedback to determine whether perceived difficulty is a limiting factor and, if so, adjusts subsequent plans to reduce difficulty or to introduce motivational scaffolds. When objective outcomes diverge from predicted trajectories, the system recalibrates RMR_combined and other physiological model parameters using inverse modeling: observed weight change over a calibrated interval is used to update energetic balance estimates and to refine the weights w_p and w_o and goal coefficients C_goal.

Data structures for storing feedback include per-action records containing the action identifier, timestamps, e_a, i_a, adherence outcome, contextual tags (for example time of day, mood, environment), and the moving averages ē_i and l̅_i. Privacy-preserving aggregation is performed to derive population-level priors for new users. Model training and personalization occur on-device, in the cloud, or in a hybrid arrangement, subject to user consent and privacy settings. Computational operations for ranking, weighting, and updating are implemented as modular services that accept standardized inputs and emit ranked intervention lists, updated target parameters, and avatar scripts for presentation.

The detailed integration of user feedback ensures that the system not only optimizes interventions for predicted physiological effect, but also adaptively learns the user's lived experience of effort and benefit. By incorporating perceived ease and perceived impact into the scoring and re-ranking mechanisms, the system increases the likelihood of adherence and of achieving user goals while maintaining transparency and user agency through conversational interactions and explicit feedback prompts.

The disclosed system and method implement a configurable, user-selectable set of lifestyle scaffolds that operate as behavioral and nutritional templates which modify tunable coefficients, constraints, and intervention priors used throughout the workflow defined by steps S100-S114. Each scaffold defines a coherent set of default parameter values for macronutrient distributions, meal timing rules, feeding windows, hydration minima, recommended food groups, permitted food exclusions, exercise timing recommendations, and goal-prioritization weightings. Selection of a scaffold causes the system to apply scaffold-specific modifiers to: (a) the resting metabolic rate estimation weighting and organ model coefficients applied in S102; (b) the temporal mapping and metabolic slope assumptions used in S104; (c) the macronutrient flow targets, hydration flows, and nourishment bottleneck heuristics used in S106; (d) the goal-specific tunable coefficients used in S108; and (e) the intervention scoring priors and feature weights used in S110. Scaffolds are offered as defaults, user-created custom scaffolds, or alternative options that the user can switch among during onboarding or during ongoing recalibration sessions initiated by the adaptive avatar interface of S114.

One exemplar scaffold is a default Mediterranean diet modification scaffold. The Mediterranean scaffold defines baseline macronutrient flow targets of approximately 40-45% calories from carbohydrates, 30-35% from fats with a focus on monounsaturated fats (e.g., olive oil, nuts), and 20-25% from protein, with tunable bands of +5% per macronutrient according to user preferences and goals. The scaffold prescribes a substantial density of whole foods, vegetables, legumes, whole grains, fish, and limited red meat, and it sets daily dietary fiber targets (e.g., 25-35 g/day) and minimum polyunsaturated:saturated fat ratios. The scaffold also provides default meal timing rules such as three regular meals with an optional one-to-two modest snacks spaced to avoid prolonged fasting beyond 12-14 hours unless the user elects intermittent fasting. Hydration flow recommendations include a per-body-mass baseline (e.g., 30-40 mL/kg/day) adjusted for environmental factors and activity. For users with cardiovascular longevity goals, the Mediterranean scaffold applies increased weight to longevity-related tunable coefficients in S108, shifting intervention ranking in S110 toward interventions emphasizing fish intake, extra-virgin olive oil, and moderate physical activity of at least 150 minutes per week.

An alternative exemplar scaffold embodies extreme carbohydrate variation options, including selectable modes such as ketogenic (very restricted carbohydrate), cyclic ketogenic (periodic carbohydrate refeeding), reduced-carbohydrate non-ketogenic, and elevated-carbohydrate athletic loading. Each extreme carbohydrate mode sets macronutrient targets and feeding timing constraints consistent with the intended metabolic state. For example, a ketogenic mode sets carbohydrate targets to 5-10% of caloric intake or absolute carbohydrate limits (e.g., 20-50 g/day), fat targets to 65-75% of calories, and protein targets to 20-25% of calories, while the cyclic ketogenic mode introduces scheduled refeed windows with increased carbohydrate intake (e.g., one day per week or targeted peri-workout carbohydrate loading). The extreme carbohydrate scaffold modifies S104 metabolic slope assumptions by increasing the expected amplitude and timing sensitivity of substrate utilization (e.g., elevated fat oxidation during fasting windows, rapid glucose utilization during refeed windows), and it alters nutrient-delivery bottleneck heuristics in S106 by accounting for glycogen repletion dynamics, ketone production thresholds, and tolerable protein intakes to prevent lean tissue catabolism.

During S100 data intake, users can explicitly select a scaffold or are presented with scaffold suggestions based on input data (age, sex, body mass, goals, workout intensity, NEAT, sleep, stress response, dietary patterns). A scaffold suggestion engine analyzes goals and behavioral inputs and computes a compatibility score for available scaffolds. Compatibility scoring is implemented as a weighted dot product of user preference vectors and scaffold priors, normalized to produce recommendation rankings. Users retain the ability to override suggestions and to customize scaffold parameters (e.g., broaden carbohydrate range from 5-10% to 10-15% for ketogenic mode).

The scaffold's influence on resting metabolic rate estimation (S102) is twofold: first, scaffolds adjust the weighting between empirical predictive equations (such as Mifflin-St Jeor or Harris-Benedict) and organ weight-based allocation models; second, scaffolds modify assumptions regarding organ-specific metabolic activity (for example, ketogenic states can induce estimated increases in hepatic ketogenesis workload and correspondingly adjust hepatic metabolic allocation coefficients). The weighted combination can be expressed as RMR=w_p*RMR_predictive+w_o*RMR_organ_model, where scaffold selection alters w_p and w_o and is configured to apply organ-specific scaling factors within the organ model.

In determining the 24-hour calorie utilization profile (S104), scaffolds inform the baseline metabolic slope function and the expected thermic effect of food (TEF) and postprandial substrate oxidation rates. For example, the Mediterranean scaffold is modeled with a moderate TEF profile and typical carbohydrate/fat/protein oxidation curves, while the ketogenic scaffold is characterized by reduced insulin-mediated carbohydrate oxidation and elevated baseline fat oxidation. Feeding and activity events are temporally aligned to the metabolic slope using scaffold-defined meal windows and exercise timing preference rules. The aligned model computes per-interval anticipated energy expenditure and substrate utilization by applying scaffold-modified coefficients to activity energy cost models and to resting slope interpolation.

Adjusting recommended targets (S108) applies the scaffold's goal-prioritization modifiers to generate final targets for longevity, general health, fat reduction, lean mass gain, and fitness. These adjusted targets are provided to the intervention scoring engine in S110. The intervention engine maintains a library of intervention features (meal substitutions, scheduling changes, exercise prescriptions, hydration increases, behavioral prompts) and a suite of scoring functions that compute additive scores, multiplicative modifiers, and coefficient-based transformations. Scaffold priors bias initial feature scores; for example, they boost scores for olive oil substitutions under the Mediterranean scaffold or elevate carbohydrate-restricted meal swaps under the ketogenic scaffold.

The visually accessible metabolic plan (S112) is rendered with scaffold-aware visualizations: pie charts reflect scaffold macro distributions, prioritized daily actions reflect scaffold-preferred behaviors (e.g., “increase olive oil”, “swap red meat for fish” for Mediterranean), and unit-specific instructions (grams, sets, minutes) map to scaffold targets. For extreme carbohydrate scaffolds, timelines explicitly indicate refeed windows and fasting windows, and quantities are provided in absolute grams and as relative percent-of-daily total.

The adaptive avatar interface (S114) presents scaffold rationale and coach-like explanations, prompts for adherence and context (e.g., “You selected the Mediterranean scaffold; would you like more plant-based fish substitutions this week?”), and supports scaffold switching with immediate recalculation. The avatar also facilitates scaffold tuning by offering incremental adjustments (e.g., narrowing carbohydrate band) and soliciting user feedback which updates scaffold parameter objects. Real-time recalibration uses recent adherence data, sensor inputs, and user-reported context to update RMR, 24-hour utilization profiles, gap quantifications, and intervention rankings; scaffold priors continue to mediate recalibration so that the delivered plan remains consistent with the user's selected lifestyle scaffold unless the user elects to change scaffolds.

Nutrient and metabolic gaps are quantified by comparing modeled supply to target flows. Caloric deficit or surplus is calculated as the difference between integrated intake energy and integrated energy expenditure over a target window (for instance 24 hours or longer). Macronutrient flow quantification tracks grams of protein, carbohydrate, and fat delivered per relevant interval and determines whether those flows meet goal-specific thresholds (for instance minimum protein for lean tissue preservation per day, carbohydrate timing relative to activity). Hydration is quantified by modeling fluid intake events against estimated fluid loss from sweat and basal losses; basic models use user-reported fluid intake and activity-derived sweat rates, while more advanced estimators incorporate environmental data and wearable sweat sensors. Nourishment bottlenecks are identified where timing or composition of feeding events reduces effective nutrient delivery to intended metabolic processes (for instance insufficient protein within anabolic windows, prolonged gaps between feeding events causing diminished amino acid availability, or elevated glycemic loads at times of reduced metabolic slope leading to inefficient substrate partitioning). The system tags intervals with shortfalls, quantifies magnitude (for instance grams of protein deficit, milliliters of fluid shortfall, caloric deficit), and estimates the impact of each shortfall on selected targets using sensitivity coefficients.

Objective=Σgw_g×f_g(targets,constraints),

Where w_g are user-weighted or system-derived weights. Constraint handling addresses user preferences (food allergies, dietary restrictions), medical constraints, and lifestyle constraints (available training time).

An adaptive avatar interface engages the user in a conversational format to present the metabolic plan, solicit adherence reports, and collect contextual updates. The avatar maintains session state and conversational context, prompting the user with targeted follow-up questions when clarifying input is needed (for example confirming meal composition, sleep disturbances, or missed workouts). Conversational interaction supports both free-text input and guided choices (quick-reply buttons) to reduce friction. The avatar monitors adherence by parsing user responses and by integrating sensor-derived adherence signals (for example corroboration of completed workouts via wearable data or logged meals via photographed receipts). Adherence metrics are computed and stored to update user profiles.

Real-time recalibration is performed in response to updated user data. Recalibration triggers include explicit user updates, sensor-derived deviations from expected behavior, periodic scheduled reassessments, and detection of significant model drift. Recalibration methods range from rule-based adjustments—for instance increasing a toward the organ model after repeated deviations—to probabilistic updating such as Bayesian updating of parameter posteriors, exponential smoothing of observed energy balance discrepancies, or Kalman-filter-like fusion of observed and predicted states. Machine learning models are retrained or fine-tuned on aggregated user-specific data to improve individual accuracy for RMR prediction, adherence modeling, or intervention effectiveness estimation. When recalibration produces materially different recommendations, the adaptive avatar summarizes the change, provides the updated action list, and explains the reason for the change.

The system architecture comprises modules for data ingestion and normalization, metabolic modeling, intervention scoring, plan generation and formatting, conversational interface management, and storage. Modules exchange information via defined APIs and message buses. The computing environment is distributed across edge devices (for latency-sensitive user interfaces and local sensor processing), backend servers (for compute-intensive model inference and storage), and external cloud services for optional model training or third-party data integration. The system maintains records of user preferences and privacy consents and provides mechanisms for data export and deletion in response to user requests. Security controls include access authentication, encryption of stored data, and transport-layer encryption for data in transit.

The system receives user-provided and sensed input data, including, without limitation, user age, sex, body mass, user-stated goals, workout intensity, non-exercise activity thermogenesis (NEAT), sleep timing and quality, stress response metrics, and dietary patterns. Input data are received through manual entry via a user interface, through connected sensors or devices (for example, wearable activity monitors, smart scales, continuous glucose monitors, or sleep trackers), via import from third-party services (for example, fitness or nutrition applications), or through combinations of these sources. Input data are normalized into a standard internal format and stored in one or more data structures. Time-stamped event logs record feeding events, activity sessions, sleep intervals, and subjective reports of stress or hunger. Default or inferred values are used for missing data, with uncertainty flags propagated through downstream computations.

RMR_organ=Σi(mass_i×metabolic_rate_per_kg_i).

The weighted combination is computed as

RMR=α×RMR_predictive+(1−α)×RMR_organ,

Where α is a tunable coefficient that can be set based on confidence in available data sources, user phenotype, or a model-selection routine. α can be adjusted automatically in response to adherence data or measured metabolic proxies (for example changes in body mass or indirect calorimetry data if provided). Confidence intervals or variance estimates associated with each model are carried forward for downstream uncertainty-aware planning.

The system is deployable on client devices, such as a mobile device displaying an avatar interface 34, or as a cloud service with client applications. The avatar interface supports multimodal interactions including text, voice, and touch, and displays environmental icons (for example, a gym building icon 38 indicating scheduled exercise) and context images (for example, a depiction of the user's hand holding the device (40)) as part of a rich user experience.

The disclosed system and method further comprise identifying and ranking the top three nutrition, movement, or sleep adjustments required to achieve selected user goals. Data received in step S100, including user age, sex, body mass, declared goals, workout intensity, non-exercise activity thermogenesis, sleep timing and quality, stress response, and dietary patterns, are processed to derive candidate adjustments and to score each candidate with respect to expected impact, feasibility, and safety. Candidate adjustments are generated by mapping gaps quantified in S106 and targets adjusted in S108 to a set of predefined intervention templates. Each intervention template corresponds to a modifiable behavior in one of the domains nutrition, movement, or sleep. Examples of nutrition templates include increasing protein intake at specific meals, redistributing carbohydrate intake toward earlier in the day, increasing total daily caloric intake or deficit by specified kilocalories, adding 200-400 mL of water at defined intervals, or introducing micronutrient-dense food items to address identified deficiencies. Examples of movement templates include adding a 20-30 minute resistance session, increasing non-exercise step count by 2,000 steps distributed across the day, inserting brief, vigorous intervals after meals to blunt glycemic excursions, or scheduling active breaks to reduce prolonged sedentary periods. Examples of sleep templates include advancing bedtime by 30-60 minutes, introducing a fixed wind-down routine of at least 30 minutes, consolidating fragmented sleep by minimizing nocturnal disturbances, and adjusting evening illumination to improve circadian alignment.

For each candidate intervention, the system computes an expected impact score by combining multiple model outputs. A primary component of the expected impact score is the marginal effect on attainment of the adjusted targets computed in S108, estimated by simulating the intervention within the 24-hour calorie utilization profile determined in S104 and the macronutrient flow models of S106. The simulation temporally aligns the intervention with feeding and activity events and computes the change in net energy balance, protein availability to target tissues, and sleep-related metabolic recovery metrics. A secondary component of the expected impact score accounts for probability of adherence, derived from historical adherence patterns present in the user data together with population priors for users with similar characteristics. A third component penalizes interventions that conflict with medical constraints or user-declared preferences.

The expected impact score E for an intervention i is represented by:

E_i=ω1*p_i*ΔT_i−ω2*C_i
Where:
   ΔT_i is the marginal effect on attainment of the adjusted targets produced by the simulation (a scalar summary of changes in net energy balance, protein availability to target tissues, and sleep-related metabolic recovery metrics),
   p_i is the estimated probability of adherence for intervention i (derived from user history and population priors),
   C_i is the penalty term for conflicts with medical constraints or user preferences,
   ω1 and ω2 are non-negative weighting coefficients used to balance the contribution of the positive effect and the penalty.

E(i)=Wimpact*SimEffect(i)+Wadherence*Padh(i)−Wcontra*ContraindicationPenalty(i)

WhereSimEffect(i) is the normalized simulated contribution of intervention i toward target attainment, Padh(i) is the predicted adherence probability, ContraindicationPenalty(i) is a binary or graded penalty for conflicts with safety rules or user restrictions, and Wimpact, Wadherence, and Wcontra are tunable coefficients that can be adjusted per goal type as described in S108.

Feasibility scores are computed for each intervention based on time cost, required resources, and user schedule compatibility. Time cost is derived from user activity calendars and typical daily routines; resource requirements are matched against user-provided environment metadata (e.g., access to a gym, kitchen equipment, or quiet sleep environment). Feasibility F(i) is normalized and combined with E(i) to form a composite priority score P(i):

P(i)=Normalized(E(i))*alpha+Normalized(F(i))*(1−alpha)

Wherein alpha is a tunable weight that reflects whether expected impact or feasibility is prioritized for the selected goal. For weight-loss and fitness targets, alpha is set to a higher value to emphasize impact; for adherence-focused or behavior-change goals, alpha is reduced to emphasize feasibility.

Safety and contraindication rules are enforced before ranking. These rules include absolute exclusions (e.g., an intervention to fast for 24 hours is excluded if medical conditions or age contraindications are present) and relative constraints (e.g., maximum recommended protein intake per kilogram body mass). When a candidate intervention triggers a contraindication, it is either removed from consideration or assigned a sufficiently large penalty in ContraindicationPenalty(i) to deprioritize it. All contraindication checks are logged for auditability and presented to the user when an intervention is excluded.

Interventions are grouped and deduplicated across domains to ensure that the top three present diverse and non-overlapping adjustments. If two top-scoring interventions exhibit considerable overlap (for example, “increase protein at breakfast by 20 g” and “increase total daily protein by 20 g”), the system merges them into a single consolidated intervention and recomputes P for the consolidated intervention. Diversity-promoting functions are applied iteratively during ranking: after selecting the current top-scoring intervention, subsequent candidate scores are discounted if they are redundant relative to already selected interventions, using a redundancy penalty R(j,k) where j is a candidate and k is a selected intervention. The adjusted score P′(j)=P(j)*(1−beta*Similarity(j,k)) where Similarity is a normalized measure of overlap and beta is a tunable parameter controlling diversity.

The top three interventions are selected by sorting candidate interventions by adjusted scores and applying tie-breaking rules. Tie-breaking prioritizes interventions with higher expected impact on the primary user-selected goal, then higher predicted adherence, then shorter required time commitment. For transparency, the system also computes an estimated time-to-impact metric for each selected intervention, an expected magnitude of change (e.g., expected daily caloric deficit in kcal, expected increase in lean tissue protein synthesis in grams per day, expected sleep consolidation measured in minutes of consolidated sleep), and a confidence value derived from uncertainty propagation through the simulation and adherence models.

User-facing presentation of the top three adjustments is generated in S112 and delivered via the adaptive avatar interface of S114. Each of the three displayed adjustments is accompanied by: a concise action statement (for example, “Add 25 g of nutrient-rich protein at breakfast”), an indicator of domain (nutrition, movement or sleep), an estimated contribution toward the target in absolute and percentage terms, an estimated time cost and recommended scheduling (specific meal or time window), a confidence/probability score, and a brief rationale linking the recommendation to the identified nutrient or metabolic gap (for example, “addresses morning protein deficit and supports lean tissue synthesis during subsequent resistance training”). Visual cues such as color coding or icons indicate priority and safety status. The user can request more detail; upon request the avatar provides an expanded rationale, alternative reduced-impact or more-feasible options, and an implementation plan with discrete steps (shopping list items, exercise set and rep prescriptions, sleep environment modifications). The display also includes an option to accept, defer, or modify each recommendation, and to set reminders or link the intervention to device sensors for automated adherence monitoring (e.g., linking a recommended post-meal walk to step counting and detecting completion).

Real-time recalibration occurs as in S114: adherence inputs, sensor-derived confirmations (e.g., accelerometry, heart rate, or sleep staging), and user feedback are processed to update adherence probabilities and observed impact. The expected impact simulations are rerun using observed adherence levels to re-estimate target-attainment trajectories. If an accepted intervention fails to produce the expected impact after an observed evaluation period, the system dynamically re-evaluates candidate interventions, re-ranks available options, and replaces one or more of the top three adjustments with alternative actions when warranted, notifying the user via the avatar and providing a concise explanation for the change.

Implementation is partitioned into modules. An input module ingests S100 data from manual entry, wearables, or third-party services. A modeling module implements S102-S106 computations and the simulation engine for intervention effect estimation. A ranking module generates candidate templates, computes E(i), F(i), and P(i), applies contraindication and diversity procedures, and outputs the top three interventions with associated metadata. A presentation module formats interventions into the visually accessible metabolic plan of S112 and the avatar-based interaction of S114. A persistence module logs interventions, user responses, and outcomes to facilitate model retraining and to provide explanations during audits. The components execute on one or more processors and can be distributed across client devices (for responsive interaction) and server-side services (for computationally intensive simulation and model updates). The system is embodied in a non-transitory computer-readable medium storing instructions that implement the modules and processes described herein.

S110 calculating a probability-ranked list of lifestyle interventions using additive, multiplicative, and weighted coefficient-based mathematical operations

RMR_combined=w_phys*Ephys+w_org*Eorg

W_phys+w_org=1, where w_phys and w_org are tunable coefficients initialized from population priors and adjusted using calibration data (e.g., measured resting energy expenditure, if available). E_org is computed by estimating organ masses from body-composition inputs or population-based scaling functions, then summing mass_i*MR_per_mass_i. The coefficients can be conditioned on user age, body composition, disease state, or longitudinal adherence, and can be updated when objective metabolic measures are obtained. Confidence intervals for RMR_combined are propagated into downstream calculations.

MR(t)=RMR_combined*C(t)+A(t)+TEF(t)+EPOC(t)

CalorieGap(t)=TargetEnergy(t)−IntakeEnergy(t)

WhereTargetEnergy(t) is derived from MR(t) and goal-modified targets (see S108). Specific nutrient gaps are computed for macronutrients and hydration. For macronutrients, the system models macronutrient flow as intake (t) convolved with a digestion/absorption kernel to produce available substrate rates to tissues. For protein, a protein availability function Pavail(t) is estimated from meal protein content, digestion rate, and anabolic windows; a muscle protein synthesis demand function Pneed(t) is derived from activity-induced sensitivity and tissue repair models. ProteinGap(t)=Pneed(t)−Pavail(t). Similar flows are computed for carbohydrate and fat for immediate oxidation versus storage, and for fluid volumes against estimated losses. Micronutrient shortfalls are identified by comparing cumulative intake against recommended daily allowances adjusted for goals and physiological status. The system detects bottlenecks, defined as intervals where one or more nutrient gaps exceed predefined or adaptive thresholds and where the timing of deficits is predicted to limit target tissue responses (for example, protein deficit during a post-resistance-exercise synthesis window).

Gap charting includes graphical representation of both caloric and specific nutrient deficits over a 24-hour slope. The rendering overlays multiple data series on a shared time axis: the metabolic rate slope MR(t), caloric intake events, activity events, and deficit series (CalorieGap(t), ProteinGap(t), CarbohydrateGap (t), HydrationGap (t)). Visual encodings use stacked area plots for cumulative deficits, line plots for MR(t) and intake, and discrete markers for events. Color coding differentiates macronutrient deficits and highlights intervals where deficits exceed threshold levels. Interactive features allow zooming to specific windows, toggling nutrient layers, and annotating causal events (e.g., skipped meal, late-night snack). The charting module computes summary statistics (total daily caloric deficit, time spent in nutrient deficit windows, peak deficit magnitude) and identifies candidate remedial actions by analyzing the timing and magnitude of deficits relative to MR(t) and tissue demand curves.

Recommended targets are adjusted (S108) by applying goal-specific tunable coefficients to base targets. A goal vector G encodes user priorities (for example longevity_weight, fat_loss_weight, lean_gain_weight, fitness_weight). Base targets such as daily caloric intake, protein grams per kg, carbohydrate distribution, and activity volume are computed from RMR_combined and MR(t) then modified:

TargetEnergy_goal=BaseEnergy*(1+alpha_G)

TargetProtein_goal=BaseProtein*(1+beta_G)

Where alpha_G and beta_G are functions of the goal vector and include constraints such as a minimum protein intake per kilogram of body weight for lean-mass gain or a maximum sustainable energy deficit for fat loss. Tunable coefficients are configurable by clinicians or set via automated personalization; different coefficient sets prioritize different tradeoffs (e.g., a longevity profile reduces protein-to-energy ratio adjustments, emphasizes glycemic stability, and shifts activity timing toward circadian alignment).

S(I)=w1*EffectivenessEstimate(I)+w2*FeasibilityScore(I)+w3*AdherenceLikelihood(I)

Where EffectivenessEstimate(I) is computed as a multiplicative model that combines expected changes in caloric balance, macronutrient availability during key anabolic windows, and exercise-induced adaptations. Probability (P_success|I) is obtained by mapping S(I) through a calibrated logistic model or by simulation. Interventions are then ranked in descending order of their probability of achieving the adjusted targets. Example interventions include strategic meal timing to supply protein during synthesis windows, a pre- or post-workout protein bolus, a targeted resistance training session to increase muscle protein synthesis sensitivity, hydration cues timed to circadian peaks, and sleep-timing adjustments to align circadian metabolism. The system optionally generates bundled interventions and computes their combined probability using dependency models to account for interaction effects.

System implementations comprise processors, memory storing program code, input/output interfaces for sensors and user devices (mobile or web), and data storage for user profiles. Data privacy and security measures are enforced in accordance with applicable standards. The system can be deployed on client devices (mobile device display with avatar), cloud infrastructure, or a hybrid arrangement, with synchronization and offline support. Algorithms are implemented as modular services for intake and preprocessing (S100), RMR estimation (S102), temporal profiling (S104), gap analysis and charting (S106), target adjustment (S108), intervention scoring and ranking (S110), plan generation (S112), and avatar-driven adaptation (S114). Example parameter values, digestion kernels, circadian factors, and coefficient initializations are stored in configurable repositories to support clinical customization and future tuning.

Recommended food portion outputs are expressed using standard hand metrics (thumb, palm, first) to improve user comprehension and adherence. The system defines baseline gram equivalents for each hand metric by macronutrient category and scales these baselines according to user-specific attributes. Baseline values can be set as follows for a reference adult (these values are illustrative and are tunable parameters in the system): thumb_fat_basis=5 g, palm_protein_basis=85 g (cooked lean protein), fist_carb_veg_basis=150 g (raw volume approximating 1 cup). For a given user, scaled values are computed:

Thumb_fat_grams=thumb_fat_basis·Ssex·Smass·Sgoal

Palm_protein_grams=palm_protein_basis·Ssex·(body_mass/70 kg)·Sactivity·Sgoal

Fist_carb_grams=fist_carb_veg_basis·Ssex·(body_mass/70 kg)·Sactivity·Sgoal

Where Ssex is a sex coefficient (e.g., 1.0 for male, 0.9 for female, or other configurable values), Smass scales with body mass relative to a 70 kg reference, Sactivity increases portion size proportional to workout intensity or non-exercise activity thermogenesis, and Sgoal adjusts portions according to the selected objective (for example Sgoal<1.0 for weight loss, Sgoal>1.0 for lean tissue gain, or configured to favor reduced carbohydrate density for longevity goals). The system supports parameter customization and clinician-specified defaults.

Converting a computed gram target g_target for a nutrient-type to hand metrics is performed by calculating the integer and fractional counts of each metric by greedy allocation or by smallest remainder rounding. For example, to express a protein target of 170 g with a palm_protein_grams value of 85 g, the system returns “2 palms.” For mixed meals, the system expresses each major component: “1 palm protein (^{~}85 g cooked), 1 first vegetables (^{~}150 g), 1 thumb fat (^{~}5 g/~1 tsp oil).” The system also supports half-palm and half-fist designations where appropriate and presents the hand metrics alongside precise gram quantities for users who prefer numerical precision.

Graphical and textual outputs are designed for clarity and actionable use. A typical daily plan entry for movement might read as a prioritized action: “Strength: Full-body session at 7:00 AM-compound lifts 3×/week; intensity 75% 1RM; 4 sets each of squat, press, row; target volume 12-16 total sets; estimated session duration 45 minutes; consume 25 g protein within 60 minutes post-session.” A HIIT example might read: “Intervals at 6:00 PM-10 rounds of 30 s effort at 90% HRmax with 90 s active recovery; total work 10 minutes; estimated calorie burn 120 kcal; follow with 15 g carbohydrate if next meal>2 hours away.” NEAT prompts can be scheduled as brief actions: “Stand and move for 5 minutes every hour” with daily cumulative targets.

Integration with wearable sensor data and manual logs refines estimation of energy expenditure and adherence. Accelerometer-derived activity counts, GPS-derived pace, and heart rate data feed the ΔE_k(t) estimates and the model of user responsiveness to different exercise modalities. When sensors are unavailable, the system falls back on user-reported activity intensity and duration, using conservative conversion factors to estimate calories.

The system prioritizes interpretable recommendations and safety. Contraindications or unusual sensor readings prompt the avatar to recommend medical consultation and to withhold vigorous exercise recommendations until medical clearance is obtained. The system stores and applies user-specific constraints and preference weights so that movement and exercise recommendations remain acceptable and sustainable for the user, thereby increasing the likelihood of adherence and improving the chances of achieving the adjusted targets.

RMR_MSJ=10*weight(kg)+6.25*height(cm)−5*age(years)+5.

For a user identified as female:

RMR_MSJ=10*weight(kg)+6.25*height(cm)−5*age(years)−161.

When sex is nonbinary or unspecified, the system applies a selected sex model, interpolates between sex-specific coefficients derived from user-provided information, or adopts a population-average formulation. The predictive-equation component is adjusted to account for recent weight change, illness, or measured metabolic markers.

The organ-system weight-based component (RMR_ORG) apportions whole-body metabolic rate across major tissues and organs using organ- or tissue-specific specific metabolic rates (kcal/kg/day) and estimated organ masses. Organ mass estimates are derived from body mass, optionally refined by measured lean mass and anthropometrics, using linear or nonlinear scaling relationships of the form:

Mass_i=alpha_i*total_body_mass+beta_i+gamma_i*(sex_indicator)+delta_i*(age_term),

Where i indexes organs/tissues such as brain, liver, heart, kidneys, skeletal muscle, and adipose tissue, and alpha_i, beta_i, gamma_i, delta_i are empirically derived coefficients. The organ system metabolic rate is computed as:

RMR_ORG=sum_i mass_i*r_i,

Where r_i is the tissue-specific metabolic rate in kcal per kg per day for organ/tissue i. The tissue-specific rates r_i and the mass-scaling coefficients are parameters stored in the system and can be tuned by population-level or individualized calibration. Typical organ/tissue categories include elevated-metabolic-rate organs (brain, liver, heart, kidneys), intermediate-rate tissues (skeletal muscle), and reduced-rate tissues (adipose), but additional subcategories can be defined where data supports finer partitioning.

A weighted combination applies a tunable coefficient w (0<=w<=1) to combine the components:

RMR_combined=w*RMR_MSJ+(1−w)*RMR_ORG.

The weight w is set by default, calibrated to the user based on available measurements (e.g., if measured resting energy expenditure is available, w is chosen to minimize error), or dynamically adjusted over time based on adherence and new data. A bias term or calibration offset can be introduced to align combined estimates with device-measured metabolic data.

Determining a 24-hour calorie utilization profile S104 models metabolic rate and event-driven energy expenditures as time-varying functions. The system defines a baseline, circadian-modulated metabolic slope M_base(t) anchored to RMR_combined and adjusted for sleep/wake timing and circadian phase. Meal-induced thermogenesis (TEF) is represented as a temporally localized elevation in metabolic rate following feeding events: for each meal j occurring at time t_j with caloric content C_j and macronutrient fractions p_protein, p_carb, p_fat, TEF_j (t)=k_j. C_j. K_TEFFilter (t-t_j), where k_j is a macronutrient-weighted TEF coefficient (protein>carbohydrate>fat) and K_TEFFilter is a decay kernel (for example, exponential or biexponential) describing the postprandial thermogenic time course. Activity events are defined by intensity and duration: ActivityEE_k (t)=MET_k·weight (kg)·K_ActivityFilter(t−t_start_k, duration_k), where MET_k is the metabolic equivalent for the activity and K_ActivityFilter allocates exercise energy expenditure across the activity interval and any transient recovery period. NEAT is modeled as a stochastic or scheduled time-varying function NEAT (t) derived from sensor data or user reports. The instantaneous total metabolic rate M_tot(t) is:

M_tot(t)=M_base(t)+E_j TEF_j(t)+E_k ActivityEE_k(t)+NEAT(t).

M_tot(t)=M_base(t)+sum_jTEF_j(t)+sum_k ActivityEE_k(t)+NEAT(t)+other_terms,

And the 24-hour calorie utilization profile is obtained by integrating M_tot(t) over the 24-hour period and by presenting the time series M_tot(t) and its cumulative integral. The metabolic rate slope is represented by the derivative dM_tot/dt or by parameterized slope terms across sleep, postprandial, and activity intervals to facilitate alignment with feeding and activity events.

Correlating feeding and activity events with the metabolic slope involves temporally aligning the time series of M_tot(t) with logged feeding timestamps and activity timestamps, enabling computation of energy balance for defined windows (for example, pre-meal, post-meal 0-4 hours, exercise+recovery windows). This alignment supports identification of periods in which feeding is mistimed relative to metabolic peaks or troughs, thereby enabling suggestions for meal timing and composition adjustments that better match metabolic demand.

Implementation details include data flow and computational considerations. Input ingestion modules validate and normalize incoming data, map categorical variables to coded representations, and store time-stamped event logs. The RMR estimation engine implements the Mifflin-St. Jeor computation and the organ-system model, provides interfaces to tune the combination weight w, and logs intermediate values for audit. The 24-hour profiling engine applies convolution operations to compute TEF and activity kernels efficiently by representing kernels in parameterized analytic form or as discrete-time filters. Gap analysis employs sliding-window aggregations and rule-based detectors to identify deficiencies and bottlenecks. The intervention ranking engine supports configurable scoring formulae, learning-based coefficient updates from outcome data, and simulation modules for joint-intervention assessment. The plan generation module formats outputs for multiple presentation modalities: text summaries for conversational exchange, graphical widgets (pie charts, timelines) for visual interfaces, and structured data for downstream export to third-party applications.

Privacy and safety mechanisms are implemented. User data is stored and processed following applicable privacy regulations. Safety constraints prevent recommendations that would exceed clinically established limits on energy deficit, macronutrient intakes, or exercise loads. The system issues warnings or directs users to consult health professionals when input data indicates medical risk (for example, extreme weight loss goals, pregnancy, or uncontrolled chronic disease).

The avatar interface presents recalibration events to the user in an actionable, empathetic manner. For example, the avatar states: “I detected that last night you went to bed 90 minutes later than usual and experienced reduced sleep efficiency. For today, I recommend reducing exercise intensity and adding two restorative actions: a 20-minute walk and a 15-minute afternoon nap. I have moved your protein-rich meal to within 45 minutes after your resistance session to support recovery.” The avatar prompts for confirmation, offers alternatives if the user cannot comply, and updates the plan instantly upon user input. If the user provides feedback indicating extenuating circumstances (e.g., travel, illness, or schedule constraints), the system adjusts recalibration parameters accordingly.

Mathematical operations used to implement recalibration and ranking include piecewise-linear scaling of recovery window duration, multiplicative adjustment of intervention scores, logistic transformations to bound scaled values, and Bayesian updating of coefficient distributions when new evidence (sensor or adherence data) is available. To convert intervention scores into user-presentable probabilities or priorities, normalized transforms such as the softmax function with a temperature parameter τ are applied; t can be reduced to produce a more decisive ranking when confidence is elevated or increased to present more balanced alternatives when confidence is diminished.

Data quality and sensor fusion considerations are incorporated into the recalibration logic. When sleep data are noisy or absent, the system falls back to multi-source estimators combining device-reported sleep with inferred sleep from inactivity periods, HRV-approximated sleep staging, and user self-reports, with corresponding reductions in confidence that affect both recalibration aggressiveness and the extent of recommendations. Privacy-preserving practices such as local computation of sensitive biometric-derived features and secure transmission of only required summary statistics are supported in exemplary implementations.

Examples illustrate typical parameterizations. In one example, wT=0.6 and wQ=0.4 are used for a user whose timing consistency historically has a larger impact on performance than single-night quality metrics. A threshold T_sleep of 0.5 (normalized scale 0-1) initiates a mild recalibration in which the recovery window is extended by 25% and an intensity multiplier for intense interventions is set to 0.8. In a second example, for an endurance athlete with wT=0.3 and wQ=0.7, a T_sleep of 0.35 triggers intervention de-escalation combined with targeted carbohydrate intake adjustments to support glycogen resynthesis during the extended recovery window.

The system supports continuous learning by storing outcomes and adherence responses to recalibration events, enabling model refinement of weighting coefficients, threshold parameters, and expected intervention effect sizes. Performance logging enables offline analysis to update population priors and personalized models. Integration with clinician or coach review flows is supported by producing exportable summaries showing sleep deviation metrics, recalibration actions, and user adherence history.

The described methods are implemented in software executing on one or more processors, in dedicated hardware, or in combinations thereof, and are operable on mobile devices, wearable devices, cloud servers, or distributed architectures. The adaptive avatar is rendered as an animated graphical agent on a mobile-device display or instantiated as a text-based conversational agent. The user interface presents the recalibrated metabolic plan as succinct daily action lists, timelines, and charts with numerical units in grams, sets, or time intervals, and enables user adjustment and obtains user consent before plan enactment.

Score_intervention=w_eff·Efficacy+w_adh·AdherenceProb−w_risk·RiskPenalty,

Where the terms are combined additively or multiplicatively and coefficients w_eff, w_adh, and w_risk are configurable. Interventions are ranked by the aggregate probability of achieving the adjusted targets, and probabilistic outputs include expected effect size, confidence intervals, and recommended sequencing.

Real-time recalibration leverages streaming data from wearables, manual entries, and third-party health APIs. The system supports asynchronous batch updates when connectivity is limited and more frequent recalibration when continuous sensor feeds are available. An adherence monitoring module assesses compliance signals, quantifies adherence rates for recommended interventions, and feeds adherence priors back into the intervention-ranking model. Where appropriate, the system proposes incremental, likely-to-succeed adjustments to improve adherence (for example, substituting readily available foods or scheduling briefer exercise bouts) and reprioritizes the three to six key daily actions accordingly.

System architecture comprises one or more processors executing modular software components including data ingestion, physiological models, intervention engine, plan generator, avatar engine, and a persistence layer. Machine-learned components optionally augment rule-based models for estimating adherence probability, sleep quality inference, stress-response interpretation, and personalized coefficient tuning. Privacy-preserving measures, such as local on-device storage of sensitive data, end-to-end encryption for communications, and configurable data-sharing consent, are supported. APIs enable clinicians or coaches to review plans, adjust coefficients, or push custom interventions when authorized.

The detailed description above is illustrative and non-limiting; alternative embodiments can vary the specific forms of the models, the selection logic for avatar type, the granularity of time intervals, and the mathematical formulations used to rank interventions, while remaining within the scope of the described system and method.

The detailed description provides examples of how the metabolic plan generated by the system includes individualized recommendations for movement and exercise, including timing, intensity, and duration, adjusted according to user goals. The system receives user-specific input data as described in S100 and uses the estimated resting metabolic rate (S102) and the 24-hour calorie utilization profile (S104) to derive exercise prescriptions that achieve the adjusted targets (S108) while respecting user constraints and preferences.

Exercise prescriptions are generated to meet one or more prioritized goals selected by the user. When the user's primary goal is weight (fat) loss, the system applies a goal-specific tunable coefficient set that biases recommendations toward a negative net energy balance while preserving lean tissue. The system computes a target caloric deficit per day and allocates the deficit between dietary adjustments and activity-based expenditure. Movement recommendations combine structured exercise (resistance training, interval or continuous aerobic sessions) and increased non-exercise activity (NEAT) to raise daily NEAT by a target ΔNEAT.

Example: For an 80 kg male with moderate workout intensity and a weight-loss goal, the system computes palm_protein_grams=85·(80/70)·1.0· 0.9≈110 g per palm (Sgoal=0.9 to reduce total caloric load while preserving protein density). With a daily protein target of 120 g, the system outputs “≈1 palm protein at breakfast (^{~}110 g) and 0.1 palm (or one reduced portion) at lunch,” and recommends “add 1 palm protein post-workout,” ranked by expected effect on lean tissue retention. The avatar schedules a reminder and, after the user reports adherence, updates available_n(t) and recalculates gaps and next-day recommendations.

The foregoing description provides enabling detail for implementing the claimed combination of metabolic estimation, time-resolved nutrient flow modeling, hand-metric based portion communication, goal-tunable targets, probability-ranked interventions, and an adaptive conversational avatar that monitors adherence and recalibrates recommendations in real time.

The detailed description provides embodiments for implementing the claimed method and system steps S100-S114, including algorithms, parameter tuning, data flows, and example calculations, and further specifies that recommendations for exercise refueling are tuned to user body weight and post-exercise metabolic requirements, with timing windows for optimal nutrient absorption.

RMRcombined=wphys*RMRphys+worg*RMRorg

Where w_phys and w_org are tunable weighting coefficients summing to 1, RMR_phys is derived from a validated predictive equation of the form:

RMRphys=a*body_mass+b*height+c*age+d*sex+e

Withcoefficients a-e chosen from published literature or empirically tuned to cohort data. RMRorg is calculated by apportioning whole-body mass into organ and tissue masses using sex- and age-specific fractionation rules, then summing organ metabolic contributions:

RMRorg=Σi(mass_i*k_i)

MRbase(t)=RMRcombined/24*C(t)

Where ∫0\hat{ }2C(t) dt=24 and C(t) is parameterized by sleep timing and chronotype. Activity events (e.g., exercise sessions) are superimposed as time-localized increments to metabolic rate computed from activity intensity, duration, and user-specific conversion factors. For each activity event j, the instantaneous metabolic increment ΔMRactivity_j(t) equals an intensity-dependent factor multiplied by body mass and metabolic equivalent (MET) scaling. Excess post-exercise oxygen consumption (EPOC) is modeled as a decaying exponential following the activity:

ΔMREPOC_j(t)=A_j*exp(−(t−t_end_j)/τ_j) for t≥t_end_j

Where A_j and τ_j depend on exercise intensity, duration, and fitness level. Feeding events are registered with caloric and macronutrient content and aligned temporally with MRbase(t) and activity events to compute interval energy balances. The 24-hour calorie utilization profile CE(t) is the sum:

CE(t)=MRbase(t)+Σ_jΔMRactivity_j(t)+Σ_jΔMREPOC_j(t)

And integrated over specified intervals to yield energy expenditure per meal and per epoch.

Target_adjusted=Target_baseline⊙(1+Cg)

Where ⊙ denotes element-wise multiplication and Target_baseline includes daily energy allowance, macronutrient gram targets, meal timing windows, hydration volume, and training load. For example, a weight loss goal increases cg_energy negative magnitude to create a caloric deficit while preserving cg_protein positive magnitude to maintain lean mass; a lean tissue gain goal raises cg_protein and cg_timing values to emphasize peri-workout protein delivery. Coefficients are tunable through user-selected aggressiveness settings and can be constrained by health safety parameters (minimum energy floors, maximum protein per kg guidelines). The adjustment step yields goal-weighted numeric targets and temporal windows for nutrient delivery.

S(li)=g(Σk wk*fik,Πm(1+vm*gim))

Wherewk are additive weights for linear effects, vm are multiplicative interaction weights for synergistic or antagonistic effects, fik are feature values, and gim are interaction indicators. The composite score is mapped to a probability P(li) via a softmax or logistic transform to produce a normalized probability distribution across interventions:

P(li)=exp(λ*S(li))/Σj exp(λ*S(lj)

Where λ controls distribution sharpness. Interventions are ranked by P(li) and presented to the user with expected impact and confidence intervals. Examples of candidate interventions include: adjust meal timing to concentrate carbohydrate immediately post-exercise; add a 20-30 g protein bolus within 1 hour post-resistance training; reduce evening carbohydrate intake; add a hydration protocol; replace a snack with higher-protein alternative; shift some caloric intake to pre-sleep protein to support overnight protein synthesis. The scoring system includes personalization via prior adherence records, stated preferences, and constraints (e.g., dietary restrictions).

Recommendations for exercise refueling are tuned to user body weight and post-exercise metabolic requirements, with timing windows for optimal nutrient absorption. The exercise-refueling module receives exercise descriptors (mode, intensity, duration, estimated glycogen depletion) and user body weight W (kg). For carbohydrates, an initial recommendation Carb_rec(t0) in grams is computed as:

Carb_rec_window=W*k_carb_base*intensity_factor*depletion_factor

Where k_carb_base is an initial tunable coefficient (example 0.5-1.2 g/kg depending on refueling aggressiveness), intensity_factor scales with relative exercise intensity (e.g., 1.0 for moderate, 1.5 for elevated intensity), and depletion_factor reflects session-specific glycogen depletion (derived from duration×intensity or from sensor-derived surrogates). Timing windows are categorized as immediate (0-2 hours post-exercise) when rapid glycogen resynthesis and enhanced glucose uptake occur, and extended (2-24 hours) when slower repletion is achievable. For scenarios requiring rapid replenishment (e.g., repeated daily training), the module recommends increased per-hour carbohydrate intake during the immediate window, for example 0.8-1.2 g/kg/hour for the first 2-4 hours, split into digestible boluses. For single-session recovery with no subsequent training, the recommendation is reduced, e.g., 0.3-0.6 g/kg in the first 1-2 hours, augmented by additional carbohydrate evenly distributed thereafter. These numeric ranges are initial tunable parameters and can be individualized by the system based on user response and safety limits.

Protein refueling recommendations target stimulation of muscle protein synthesis and repair. A protein recommendation Prot_rec is computed as:

Prot_rec_meal=W*k_prot_meal

Wherek_prot_meal is a per-kilogram coefficient typically in the range of 0.25-0.4 g/kg (example values: ^{~}0.25-0.4 g/kg per feeding for maximal acute muscle protein synthetic response). The post-exercise timing window for maximal effect is typically within 0-2 hours post-exercise. For example, for a 70 kg user, Prot_rec_meal yields approximately 17.5-28 g protein immediately post-exercise. For extended recovery, the system sequences protein feedings at intervals (e.g., every 3-4 hours) to maintain elevated amino acid availability for 24 hours post-exercise. Protein quality (leucine content, digestibility) is included as a modifier to reduce recommended grams when a protein source of superior composition is selected.

Hydration refueling is tuned to estimated fluid losses L (liters) derived from pre- and post-exercise body mass changes and sweat-rate models. The rehydration volume Hydr_rec is calculated as:

Hydr_rec=L*r_repl

Where r_repl is a repletion factor (e.g., 1.25-1.5) that accounts for ongoing losses and incomplete absorption. Sodium and electrolyte recommendations are adjusted based on exercise duration and estimated sweat sodium to optimize fluid retention and absorption. For prolonged or elevated-temperature activities, electrolyte-enriched fluids are recommended within the immediate 0-4 hour window.

The exercise-refueling module includes decision logic that prioritizes refueling actions based on proximity to the next training session, magnitude of glycogen depletion, and user goals. When the next training session is within a brief interval (e.g., <8 hours), the system elevates carbohydrate urgency and recommends partitioning carbohydrate intake into multiple modest, rapidly absorbed boluses during the proximal window. When the next session is remote (>24 hours) and the goal emphasizes fat loss, the system is configured to recommend a more conservative carbohydrate intake while ensuring adequate protein to preserve lean mass.

Timing windows and absorption kinetics are represented with digestion and absorption transfer functions that convert ingested macronutrient grams into circulating substrate availability over time. The system employs meal matrix templates with empirically derived absorption half-times (e.g., rapid liquid carbohydrate ^{~}30-60 minutes; mixed solid meals longer) and adjusts recommendations to align expected availability with metabolic demand CE(t). For example, to match an intense interval training session ending at t_end, a carbohydrate bolus scheduled at t_end+10-30 minutes is preferred to maximize insulin-mediated glycogen uptake, whereas a mixed solid meal is scheduled at t_end+60-90 minutes.

Adherence monitoring for refueling recommendations is enabled by prompting users to confirm intake, scanning meal images to estimate portions, integrating with logged meals, and using weight and glucose responses as proxy feedback. After confirmed refueling, the system recalculates the nutrient gap and, if deficits persist, elevates the intervention ranking to recommend additional incremental administrations or alternative formats (liquid shakes, gels, easily digestible snacks). The system communicates the confidence and expected efficacy of each recommended refueling action to the user as probability metrics and presents alternative reduced-intensity and higher-intensity options.

Representative parameter values and example computations are provided to illustrate operation. For example, for a 35-year-old male, 80 kg, height 180 cm, the predictive RMR might compute 1,800 kcal/day, the organ-weight model might compute 1,650 kcal/day, and with α=0.6 the combined RMR would be 1,740 kcal/day. Diurnal modulation might apply a ±5% circadian amplitude peaking in the early evening. An afternoon 45-minute run at 8 METs would be converted to AEE≈(METs−1)×3.5×body_mass_kg/200×duration_minutes, producing an interval expenditure that is inserted into the 24-hour profile. If intake for the day is 2,200 kcal, the net balance against a modeled 2,650 kcal integrated expenditure yields a 450 kcal deficit; the system then identifies protein flow shortfalls (for example total protein 70 g versus target 120 g) and ranks interventions such as “add 30 g protein at breakfast” with an estimated probability of preserving lean mass derived from the intervention scoring function. Iterative daily recalibration uses observed weight trends and logged intake to adjust underlying model coefficients and to update the α parameter if the predictive model consistently over- or under-estimates energy needs.

Embodiments include computer-readable media storing instructions that cause processors to perform the described method steps and systems comprising processors, memory, and user interfaces configured to execute the method. The described features are combinable in various permutations, and the method is implementable in software, firmware, hardware, or any combination thereof. Variations include alternative predictive equations, different organ partition schemes, alternative scoring functions, and alternate conversational modalities. The described examples illustrate the operation and do not limit the scope of potential implementations.

Selection of the predictive equation is performed dynamically based on BMI and reported activity levels. For example, the system evaluates BMI and an activity score A (derived from reported workout intensity, frequency, and wearable-measured activity) and applies selection logic such that: if BMI is below an underweight threshold (e.g., BMI<18.5) the system favors equations that perform well in reduced body-mass contexts; if BMI falls within a normal range (e.g., 18.5≤BMI≤30) the system favors general-purpose predictive equations such as Mifflin-St Jeor; if BMI exceeds an elevated threshold (e.g., BMI>30) the system prefers equations or corrections that adjust for adiposity and that can be combined with organ model outputs to mitigate known biases. Activity-driven selection additionally routes users with substantial resistance-training exposure or greater measured lean-mass estimates to lean-mass-based equations (for example, Cunningham or Katch-McArdle), whereas sedentary users are routed to population-average predictive equations. Selection can be implemented as threshold-based routing, probabilistic model mixing, or via a trained classifier that outputs weights for each available predictive equation conditioned on BMI, activity score A, age, sex, and available body composition data.

The final resting metabolic rate estimate R_RMR is computed as a weighted combination of the selected predictive equation output R_pred and the organ model output R_org: R_RMR=α·R_pred+(1−α)·R_org, where α is a tunable weighting coefficient in the interval [0,1]. α can be predetermined by the selection logic, learned from population calibration data, or adapted on a per-user basis according to feedback signals (for example, measured weight change versus predicted energy balance, or indirect calorimetry when available). The system supports time-varying a to reflect changes in body composition or measurement confidence.

Determining a 24-hour calorie utilization profile (S104) comprises constructing a temporally resolved model MR(t) for t in a 24-hour cycle that maps baseline resting metabolic rate to hourly or sub-hourly expected metabolic rates and overlays event-driven energy expenditures. MR(t) is computed as:

MR(t)=R_RMR·C_circ(t;φ,A_circ)+Activity_EE(t)+TEF(t)+NEAT_adj(t),

Where C_circ is a circadian scaling factor parameterized by phase φ and amplitude A_circ (for example C_circ(t)=1+A_circ·sin (2π(t−φ)/24)), Activity_EE(t) represents activity-induced energy expenditure computed from event records as Σ_j MET_j·M_body·duration_j (METs converted to kcal/min using body mass), TEF(t) is the thermic effect of feeding temporally aligned with feeding events and estimated from macronutrient composition (protein TEF>carbohydrate TEF>fat TEF; TEF magnitude and time course are modeled as functions of meal calories and macronutrient percentages), and NEAT_adj(t) is a time-dependent NEAT contribution inferred from device motion and self-report. Activity_EE and TEF are temporally correlated with user-logged or sensor-detected events; each feeding event generates a TEF waveform with configurable rise and decay parameters, and each activity event generates an activity EE waveform based on type, intensity, and duration. The system computes the slope of MR(t) across administration intervals and identifies intervals of rapid change, plateaus, and troughs for use in nutrient flow and intervention design.

Bottleneck identification uses constraint analysis: a bottleneck is declared when an upstream constraint (for example, insufficient protein during an anabolic window, inadequate carbohydrate during prolonged, intense activity, or insufficient fluid intake during heat exposure) prevents meeting tissue-level targets. Hydration gaps are quantified by comparing estimated sweat losses and fluid intake to hydration targets scaled to activity duration and exertion level.

Outputs include cumulative daily deficits, temporal localization of deficits, and minimal corrective interventions required to resolve identified bottlenecks.

Implementation is realized as software modules executing on general-purpose processors, distributed across mobile devices and cloud servers, and utilizing secure data stores for user state and model parameters. The system supports parameter tuning, clinician-configurable coefficient templates, and machine-learning pipelines that refine selection logic, weighting coefficients, and scoring parameters using aggregated, anonymized outcome data. The described methods enable individualized, temporally resolved metabolic planning that dynamically adapts predictive-equation selection based on BMI and activity levels and integrates organ-system modeling, event-aligned energy expenditure, nutrient-flow analysis, goal-weighted target adjustments, probability-ranked interventions, and an adaptive conversational avatar to guide user behavior.

Protein flow timing bottlenecks are identified by comparing per-meal and per-window protein delivery against goal-specific protein-flow targets. Target total daily protein P_total is computed as a function of body mass, sex, age, and goal-specific multipliers (for example P_total=β_goal·mass, where β_goal ranges from approximately 0.8-2.4 g/kg/day depending on sedentary versus aggressive lean tissue gain goals). Per-meal target P_meal_target is computed to maximize muscle protein synthesis and to avoid prolonged periods of insufficient amino acid availability; example targets are on the order of 0.3-0.45 g/kg per meal for anabolic goals and 0.25-0.35 g/kg per meal for maintenance. The system flags a protein-flow bottleneck when either (a) inter-meal intervals exceed a threshold (for example>5-6 hours), producing prolonged amino acid scarcity relative to T_n(t), or (b) a majority of daily protein is consumed in a single feeding event such that per-meal distributions are below P_meal_target for two or more meals. User-specific recommendations are generated for each identified protein bottleneck, such as reorganizing intake to distribute protein more evenly across meals (e.g., shifting 10-20 g from a late meal to breakfast), recommending specific food swaps (e.g., adding 20-30 g of Greek yogurt or 30-40 g cooked lean meat), prescribing a peri-workout protein dose (e.g., 20-30 g whey or mixed protein within 60 minutes of exercise), or suggesting supplement timing and protein type based on digestion kinetics and user preference. Recommendations include precise units (grams per meal), timing windows, and optional recipes or packaged-food suggestions to facilitate adherence.

Carbohydrate distribution bottlenecks are identified by evaluating carbohydrate availability relative to predicted energy demands, glycemic control objectives, and workout timing. The system computes per-window carbohydrate targets C_target(t) to support training sessions and to modulate insulin exposure for metabolic goals. A carbohydrate bottleneck is flagged when observed carbohydrate supply O_c(t) is insufficient during elevated-demand windows (for example pre-, intra-, or post-exercise), or when carbohydrate load is concentrated at metabolically suboptimal times (for example very late-night pronounced glycemic loads compromising sleep and overnight fat oxidation). User-specific recommendations include shifting carbohydrate to peri-workout windows (e.g., 25-60 g of moderate-glycemic carbohydrate 30-60 minutes pre-exercise for moderate-duration workouts, or split dosing of 30-90 g/hour during prolonged workouts), reducing late-evening pronounced glycemic loads by substituting options with decreased glycemic index, and distributing total daily carbohydrates into meals sized to meet both energy and glycemic targets (grams per meal specified). For users targeting fat loss, carbohydrate timing recommendations emphasize aligning increased carbohydrate loads with periods of elevated metabolic slope or workout windows, while decreasing carbohydrate magnitude during periods of minimal activity.

Omega-3, omega-6, and omega-9 fatty acid ratio bottlenecks are identified by analyzing reported dietary fat sources and, if available, supplement usage. The system computes estimated intake of EPA/DHA and alpha-linolenic acid (omega-3), linoleic acid (omega-6), and oleic acid (omega-9/MUFA) from known food composition tables. A ratio metric R_6_3=(omega-6 intake)/(omega-3 intake) is computed and compared against goal-adjusted targets; typical target ranges set R_6_3≤4:1, with narrower targets (≤2:1) for cardiovascular or inflammatory-prone phenotypes, and greater tolerance for certain cultural diets while recommending progressive adjustment. Absolute omega-3 intake targets are also computed (for example targeting≥250-1000 mg/day combined EPA/DHA for general health to clinical indications depending on goals). A bottleneck is flagged if R_6_3 exceeds the target, if absolute omega-3 intake is below target, or if omega-9 intakes are disproportionately insufficient relative to desired MUFA proportions (for example MUFA target 15-25% of calories). User-specific recommendations include adding fatty fish servings (e.g., 1-3 servings/week with a target 250-500 mg EPA/DHA per serving estimated), introducing plant sources of omega-3 or recommending concentrated supplements (e.g., 500-1000 mg EPA+DHA/day where appropriate), substituting linoleic-rich oils with oleic-enriched or MUFA-rich oils (e.g., olive oil, sunflower oil enriched in oleic acid), and specific food swaps (e.g., replacing three tablespoons per day of soybean oil with olive oil). Recommendations specify quantities (e.g., grams of oil, grams of fish, supplement mg), timing (meals to include omega-3-rich sources), and progressive schedules to gradually re-balance ratios while considering user preferences and adherence constraints.

Vegetable intake bottlenecks are identified by comparing reported servings, fiber grams, and phytonutrient diversity to target profiles tailored to goals and health status. Vegetable targets are computed as a combination of total servings per day (for example 3-10 servings/day depending on goals), fiber targets (for example 25-40 g/day for adults, adjusted for sex and age), and diversity metrics (e.g., inclusion of leafy greens, cruciferous vegetables, and colored vegetables across the week). Bottlenecks include insufficient total servings, inadequate fiber, or insufficient diversity that can impair micronutrient and microbiome-mediated benefits. User-specific recommendations include adding specified vegetable servings (e.g., +1 cup raw leafy greens at breakfast, +½ cup cruciferous vegetable with lunch), providing precise measures in grams or cup-equivalents, recommending practical preparation methods, integrating vegetables into preferred recipes, and suggesting timing to improve satiety and postprandial glycemia (for example consuming non-starchy vegetables earlier in meals). Recommendations also include swapping or supplementing with concentrated sources (e.g., fermented vegetables) when fresh intake is constrained.

The system applies goal-specific tunable coefficients (S108) to weight the relative importance of each nutrient axis and timing. These coefficients influence target calculations (β_goal for protein, carbohydrate timing multipliers, ratio tolerances for fatty acids, vegetable serving priorities) and are adjustable through clinician input, user preference settings, or automated learning from adherence and outcome data. For example, in a lean tissue gain profile the system increases the coefficient on protein flow timing and peri-workout carbohydrate delivery, whereas in a longevity-focused profile the system increases the coefficient assigned to vegetable diversity and the omega-3 ratio.

A probability-ranked list of lifestyle interventions is computed (S110) by scoring potential interventions according to expected impact on gap scores, feasibility, and predicted adherence. Scoring functions combine additive and multiplicative operations: score_i=w_impact·Efficacy_i+w_adherence·AdherenceLikely_i−w_cost·Burden_i, where Efficacy_i is an estimated reduction in G_n if intervention i is implemented, AdherenceLikely_i is a probabilistic estimate based on user history and population priors, Burden_i quantifies time/cost complexity, and w_* are tunable weights. Interventions addressing identified bottlenecks (protein timing adjustments, carbohydrate timing changes, omega-3 supplementation or food swaps, explicit vegetable-serving additions) are prioritized and ranked. Each recommended intervention is accompanied by metadata: expected reduction in specific gap scores, confidence intervals, units for implementation (grams, servings, mg supplementation, timing windows), and suggested monitoring metrics.

A visually accessible metabolic plan is generated (S112) that conveys the prioritized interventions as three to six key daily actions, graphical summaries (such as pie charts of macronutrient flows and food groups), and detailed units (grams protein per meal, carbohydrate grams per peri-workout window, omega-3 mg/day targets, vegetable servings and grams). Example actionable daily actions might include: 1) consume 30 g protein within 60 minutes of waking and 30-35 g protein within 60 minutes post-exercise; 2) shift 40-60 g carbohydrate to 30-60 minutes pre-exercise on training days; 3) add two servings of mixed vegetables at lunch and one serving at dinner totaling 300-400 g; 4) replace cooking oil rich in linoleic acid with olive oil (20 g/day) and add a 500 mg EPA+DHA supplement at dinner. The plan includes timelines, meal-level breakdowns, and optional recipes or shopping items to facilitate execution.

Example implementation details include discrete-time algorithms for flow computation, threshold parameters for bottleneck detection, and user-interface templates. A sample protein-bottleneck detection pseudocode, executed over a 24-hour discretized timeline, is as follows: compute P_total_target; compute P_meal_targets for n meals/windows; for each window compute O_p(window); if O_p(window)<P_meal_target AND sum of deficit windows>δ_protein_flag then flag a bottleneck and generate recommendation set R_protein consisting of meal swaps, specific gram targets, and timing windows. Similar code patterns apply to carbohydrate, fatty-acid ratio, and vegetable intake analyses. Thresholds δ_* and target ranges are stored as adjustable policy parameters and are configurable according to clinician or user preference.

The system supports iterative validation and enablement by supplying rationales with each recommendation, quantifying expected benefit (for example percent reduction in protein gap, expected increase in time-in-range for glycemia), and providing monitoring metrics (e.g., weekly check-ins, weight/fat/lean mass trends, symptom logs). In alternate embodiments clinician review layers, safety checks for interacting conditions (e.g., renal impairment limiting protein dosing), and regulatory disclaimers are incorporated. The disclosed methods and system enable practical, personalized identification of dietary timing and composition bottlenecks and generation of concrete, measurable, and user-specific recommendations targeted to optimize metabolic outcomes across differing goals.

The method further comprises dividing daily recommended macronutrient intake by the user's typical meal frequency to estimate per-hour or per-meal nutritional targets. Daily targets for protein, carbohydrate, and fat are calculated from adjusted recommended targets (S108) and then partitioned according to the user's habitual meal frequency and timing. For instance, if daily protein target P_daily is 120 grams and the user reports three principal meals per day with one small snack, the system can compute a baseline per-meal distribution such as P_meal=P_daily/3=40 g for primary meals, and P_snack=residual allocation (for example 0-10 g depending on user preference). Alternatively, the system can compute per-hour targets for users consuming multiple small meals or employing time-restricted feeding by dividing P_daily by the number of feeding hours or by applying a continuous per-hour delivery curve that concentrates intake around training times or circadian anabolic windows. The per-meal or per-hour allocation can be further adjusted by activity timing—for example increasing protein allocation in the feeding window immediately following a resistance training session to meet elevated muscle protein synthesis demands. The partitioning logic supports uneven distributions and user-configurable rules so that specific meals (e.g., breakfast, post-workout) can receive prioritized macronutrient allocations. Examples: with P_daily=120 g and meal frequency n=3, baseline P_meal=40 g; with n=6, P_meal=20 g. If a user prefers protein-centric pre/post-workout nutrition, the system can assign +25-50% of baseline to the post-workout meal and reduce other meals proportionally.

Implementation details include storing user data, model parameters, and historical logs in structured databases; executing computations on local device processors or remote servers; and presenting results through a graphical user interface that can be deployed on mobile devices, web platforms, or integrated devices. The system is configured to integrate with wearable devices and third-party data sources via APIs to collect activity, heart rate, sleep, and contextual data to improve real-time estimates. Security and privacy controls are applied to protect health-related user data.

Example workflows illustrate the system operation. A 35-year-old male, 80 kg, aiming for lean tissue gain reports three meals and one post-workout snack per day, resistance training at 17:00, and average sleep from 23:00 to 07:00. The system computes an RMR via the weighted combination (S102), models a daily metabolic slope with postprandial and post-exercise elevations (S104), computes a protein target of 150 g/day adjusted for lean gain (S108), divides this by meal frequency to assign 40 g to each primary meal and 30 g to the post-workout snack, and recommends prioritized interventions such as increasing post-workout protein and slight caloric surplus around training (S110-S112). The avatar (S114) confirms ingestion of the post-workout snack and, receiving an affirmative, reduces the probability weight on alternative interventions and updates the nutrient gap quantification accordingly.

The described embodiments are illustrative and permit numerous variations, including different predictive RMR equations, alternative organ system parameterizations, adaptive learning of coefficients from cohort performance, varied partitioning heuristics for per-meal allocations (equal, weighted by user preference, or activity-proximal weighting), and differing scoring architectures for intervention ranking. The per-meal and per-hour partitioning logic can incorporate constraints such as maximum practical meal sizes, digestion limits, and user food preferences to produce feasible and personalized execution plans.

Candidate interventions are defined as discrete, actionable modifications to behavior, nutrition, hydration, sleep, or exercise that are predicted to move the user toward one or more adjusted targets. Each candidate intervention i is represented by a feature vector F_i containing measurable or model-predicted attributes including, but not limited to, estimated incremental energy balance change ΔE_i(t) over specified intervals, macronutrient flow modifications ΔM_i, timing alignment with the user's metabolic slope, expected effect on hydration H_i, expected effect on sleep quality S_i, compliance cost C_i (time, perceived difficulty), and interactions with identified tissue-specific nutrient bottlenecks B_k. Feature extraction populates F_i from the user data and submodels produced in steps S100-S106 and S108.

The additive component A_i of an intervention score aggregates positive contributions from features that directly support target achievement. A general form of the additive component is:

A_i=Σ_j w_j*φ_j(F_i,j)

Where F_i,j denotes the j-th feature of intervention i, φ_j(·) is a feature-specific transformation or normalization function (for example, scaling ΔE_i to kcal/day units or converting sleep improvement S_i to a 0-1 scale), and w_j is a tunable, goal-specific coefficient that weights the relative importance of feature j for the active user goal(s). Examples of φ_j include linear normalization to unit range, piecewise linear reward for within-target macronutrient windows, or time-weighted contributions for actions that align with circadian metabolic slope. The set of w_j coefficients is selected according to the adjusted targets produced in S108; distinct coefficient sets are maintained for different goals (e.g., fat loss, lean tissue gain, longevity), allowing the additive aggregation to reflect goal-specific priorities.

Bottlenecks and limiting conditions identified in S106 are encoded as multiplicative penalty factors. Each bottleneck k associated with the user (e.g., chronic hydration deficit, micronutrient limitation at target tissue, misaligned feeding relative to peak metabolic slope, elevated perceived stress) is described by a severity metric b_k in the interval [0,1], where 0 denotes absent and 1 denotes severe. For each intervention i, a per-bottleneck sensitivity parameter s_{i,k}∈[0,1] reflects how much the presence of bottleneck k reduces the efficacy of intervention i. The multiplicative penalty P_i is computed as: P_i=Π_k (1−s_{i,k}·b_k).

P_i=Π_k(1−p_k*s_{i,k}*b_k)

The un-normalized composite score S_i for intervention i is obtained by combining the additive gain and the multiplicative penalty. A representative formulation is:

S_i=(A_i+ε)*P_i

Where ε≥0 is a minimal baseline constant that ensures nonzero scores for interventions whose additive components are near zero yet remain acceptable or incur modest costs. Alternative formulations include an optional multiplicative scaling to penalize interventions with substantial compliance costs, e.g. S_i=(A_i+ε)*P_i*(1−γ*C_i_norm), where C_i_norm is a normalized compliance cost and γ∈[0,1] down-weights costly actions.

To convert raw scores into a probability-ranked list, the scoring engine maps the set of S_i values to probability-like weights via a normalization operation that preserves relative magnitude and provides interpretable metrics. Two common mappings are softmax normalization and a logistic mapping to an independent probability of success. For softmax ranking across candidate set {i}, probabilities are computed as:

Pr_i=exp(τ*S_i)/Σ_j exp(τ*S_j)

Where t≥0 is a temperature parameter controlling the spread of the distribution; larger τ emphasizes top-scoring interventions. For independent-probability estimation, each S_i is passed through a sigmoid function:

Pr_i=1/(1+exp(−α*(S_i−β)))

Where α controls slope and β sets a midpoint; α and β are tunable or learned to calibrate Pr_i against observed outcomes. The system can employ cross-validation on historical user response data to select τ, α, β and other coefficients such that predicted probabilities align with empirical success rates.

Expected impact estimates accompany Pr_i by simulating the effect of the intervention on adjusted targets over user-specific time horizons. For each intervention i, the engine computes an expected target delta E[ΔT_i] using submodels of energy balance and tissue kinetics derived from S102-S106. For instance, expected fat mass change is estimated by converting predicted cumulative energy balance change into adipose tissue mass using an energetics-to-mass conversion factor that depends on user physical parameters; expected lean mass change is modeled using anabolic stimulus-to-fat-free-mass transfer functions modulated by protein intake and resistance training intensity. These expected impacts are presented alongside Pr_i to provide both a likelihood and a magnitude dimension for decision-making.

Uncertainty quantification is produced by propagating uncertainty in input measurements, model parameters, and compliance estimates through the scoring and simulation pipeline. Monte Carlo sampling or analytic variance propagation yields confidence intervals for S_i, Pr_i, and E[ΔT_i]. Interventions whose confidence intervals straddle thresholds for clinical or user-notable change are flagged for prioritized monitoring by the adaptive avatar interface. The avatar is configured to request brief adherence or outcome feedback after a preset interval, and that feedback is used to update bottleneck severities b_k, tunable coefficients w_j and p_k, and sensitivity parameters s_{i,k} via Bayesian updating or online learning routines, enabling real-time recalibration (S114).

The scoring engine is extensible to account for interaction effects among concurrently recommended interventions. When a set of interventions {i, j, . . . } are to be evaluated jointly, pairwise interaction coefficients ρ_{i,j} capture synergy (ρ>0) or antagonism (ρ<0). Joint score S_{i,j, . . . } can be computed as:

S_{joint}=(Σ_i A_i+Σ_{i<j}ρ_{i,j}*InteractionTerm_{i,j}+ε)*Π_k(1−p_k*CombinedSensitivity_k*b_k)

Here, InteractionTerm_{i,j} represents the expected incremental benefit or cost when interventions i and j are combined, and CombinedSensitivity_k captures how joint interventions are influenced by bottleneck k. The ranking algorithm can propose individual interventions or combinations of limited cardinality as candidate interventions, constrained by computational budget and user-preference restrictions.

The system enforces safety and plausibility constraints during scoring. Hard constraints block interventions that would violate clinician-specified limits (for example, minimum daily energy intake or maximum allowed change in sodium intake), and soft constraints apply additional penalties in the multiplicative penalty term. The scoring engine records the rationale for each penalty and the contributing bottlenecks to enable transparent explanation to the user via the avatar. Explanations include which bottlenecks are limiting an intervention, how the penalty magnitude was derived, and what upstream actions can reduce the bottleneck to restore efficacy.

Parameter initialization and ongoing tuning of coefficients (w_j, p_k, s_{i,k}, τ, α, β, ρ_{i,j}) are performed using a combination of domain knowledge, population-level training data, and user-specific calibration. During initial deployment, priors for coefficients are seeded from published clinical guidance and physiological literature. As user-specific adherence and outcome data accumulate, the system utilizes regularized maximum likelihood estimation or Bayesian posterior updating to refine coefficients, balancing responsiveness to individual data with stability derived from population priors. The system is configured to support federated update modes whereby aggregated, anonymized summary statistics improve priors without sharing raw user data.

The adaptive avatar interface (S114) leverages the probability-ranked list to drive conversational decision-making. The avatar presents the top N interventions with concise statements of Pr_i, expected impact, and primary bottlenecks, and solicits user preferences or constraints. User responses—affirmations, rejections, or contextual constraints—are fed back to the scoring engine, which re-scores and re-ranks interventions in real time. The avatar also schedules lightweight micro-feedback prompts after an intervention has been initiated; observed adherence and measured outcomes are used to update b_k and the coefficients, closing the loop for continuous, dynamic personalization.

Implementation details include storage of candidate intervention templates, feature extraction pipelines, and coefficient sets within a non-transitory memory; execution of feature transformations, score computation, and normalization on one or more processors; and exposure of the ranked list and explanatory metadata via the user interface. To support reproducibility and auditability, the engine logs input data versions, coefficient values used, intermediate A_i and P_i values, and any safety overrides applied during each ranking computation. This log can be used for offline model retraining and for generating user-facing summaries of why particular interventions were recommended or deprioritized.

Example numeric illustration: suppose a candidate intervention i is “increase protein intake at breakfast by 20 g and perform 10 minutes of resistance band work post-breakfast.” Extracted features yield ΔE_i=−25 kcal/day (net energy change after accounting for increased thermic effect), ΔM_i (protein) normalized=0.8, timing alignment=0.9 (aligns well with morning metabolic slope), H_i=0 (no hydration change), and C_i_norm=0.2. The additive coefficients for the active goal (lean tissue gain) are w_energy=0.4, w_protein=0.35, w_timing=0.25. Using linear φ transforms, A_i=0.4*(−25 normalized to −0.1)+0.35*0.8+0.25*0.9≈−0.04+0.28+0.225=0.465. Suppose a single bottleneck exists, b_1=0.6 (moderate gut absorption issue), with p_1=0.5 and s_{i,1}=0.8, then P_i=1−0.5*0.8*0.6=1−0.24=0.76. With ε=0.01, S_i=(0.465+0.01)*0.76≈0.355. Applying a sigmoid with α=5 and β=0.2 yields Pr_i≈1/(1+exp (−5*(0.355−0.2)))≈0.71. The interface could therefore present this intervention with an estimated 71% likelihood of helping achieve the lean mass target and note that gut absorption is limiting efficacy, offering upstream fixes (e.g., modest fiber adjustment, probiotic trial) that, if addressed, would increase P_i and hence Pr_i.

The described scoring architecture enables interpretable, tunable, and adaptive ranking of lifestyle interventions by explicitly separating additive beneficial effects from multiplicative bottleneck penalties, providing both probability estimates and expected impact magnitudes, and supporting real-time recalibration through the adaptive avatar interface. The method is compatible with various submodels for energy expenditure, nutrient kinetics, and behavioral adherence, and is designed to be extensible as additional bottlenecks, interaction effects, or intervention categories are identified.

Embodiments provide a system and method that collect and process user-specific input data (S100), including age, sex, body mass, expressed goals, workout intensity, non-exercise activity thermogenesis (NEAT), sleep timing and quality, stress response indicators, and dietary patterns from one or more sources such as manual user entry, wearable sensors, mobile device sensors, and third-party activity or nutrition services. The received input data are normalized, time-stamped to a common timeline, and stored in a user profile data structure. Historical records and recent windows (e.g., past 7, 14, and 30 days) are maintained to enable trend calculation and to provide baselines for deviation detection and model recalibration.

Real-time recalibration of recommendations is performed when deviations in sleep timing or sleep quality are detected. Sleep deviations are detected by comparing incoming sleep timing and quality metrics from sensors or self-report to expected or baseline values stored in the user profile. Sleep timing deviation ΔT is calculated as the absolute difference between actual sleep onset/offset times and target or median times. Sleep quality deviation ΔQ is derived from one or more metrics such as sleep efficiency, total sleep time, rapid eye movement (REM) proportion, slow-wave sleep proportion, or autonomic markers such as nocturnal heart rate variability (HRV). A composite sleep deviation score S_sleep is computed as a weighted sum or other combination, for example S_sleep=wT·normalize(ΔT)+wQ·normalize(ΔQ), where wT and wQ are tunable weights that reflect the relative importance of timing versus quality for the user or goal.

When S_sleep exceeds a predefined threshold T_sleep, the system triggers a recalibration routine that modifies the recovery window and intervention priority accordingly. The recovery window is represented as a time interval (or set of intervals) following activity in which the system anticipates enhanced restorative processes and during which certain interventions are recommended or deprioritized. Recalibration increases, decreases, or shifts the recovery window duration and timing in proportion to the magnitude and direction of the sleep deviation. For example, a delayed sleep onset of one to two hours with moderate quality degradation results in pivoting the recovery window later by one hour and extending its duration by 20-50%, whereas severe sleep fragmentation causes the system to extend recovery periods by a larger factor and to reduce recommended intense interventions for the succeeding day(s).

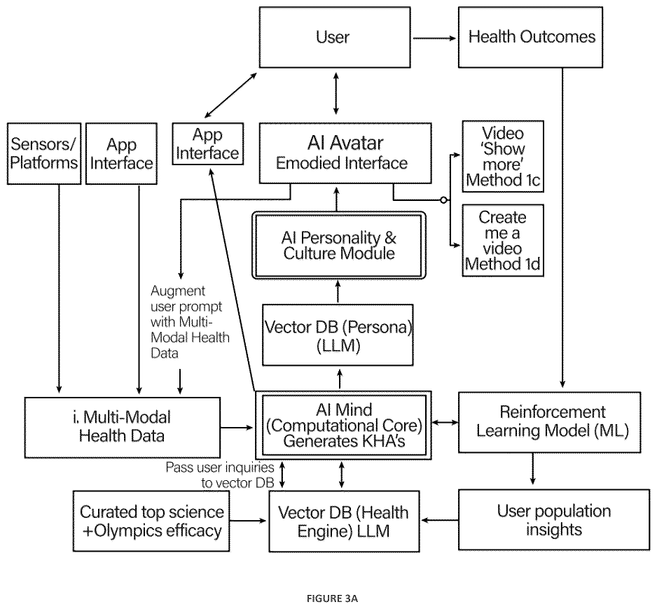

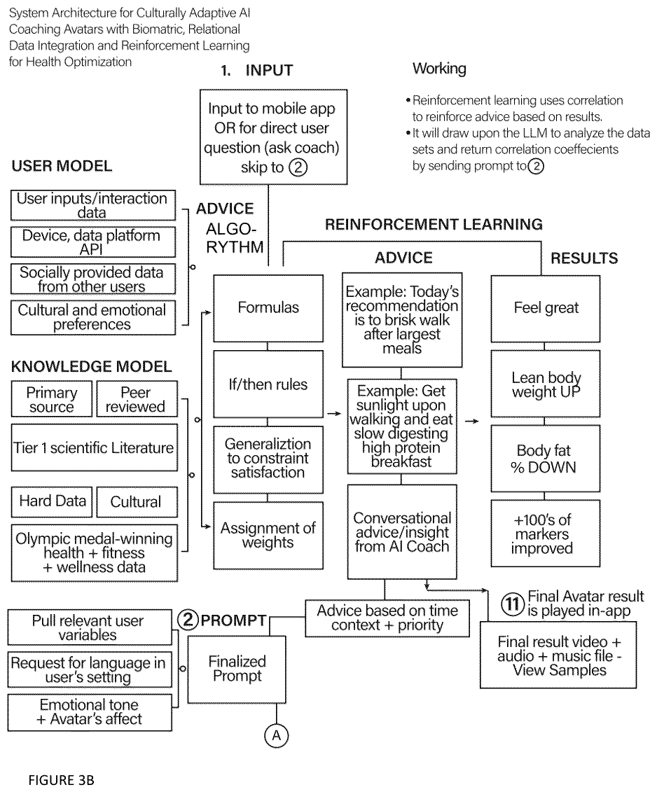

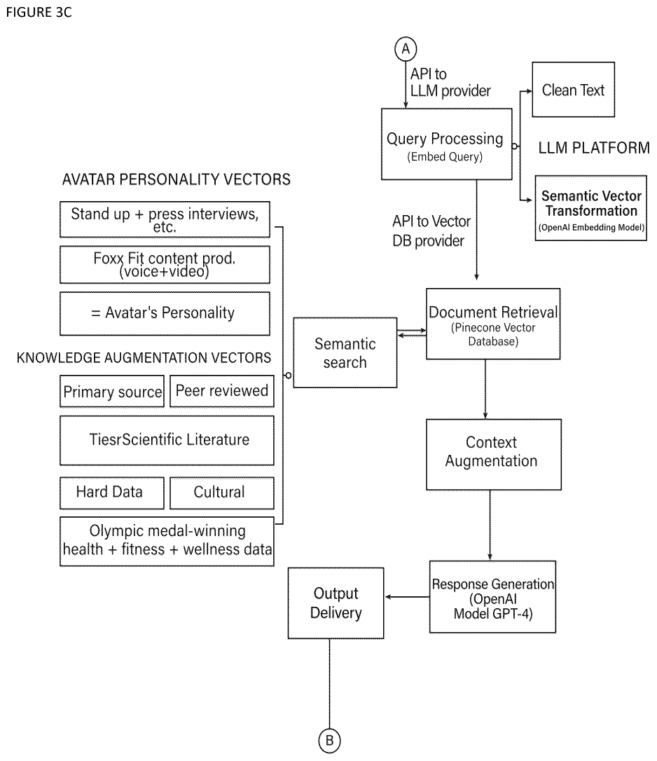

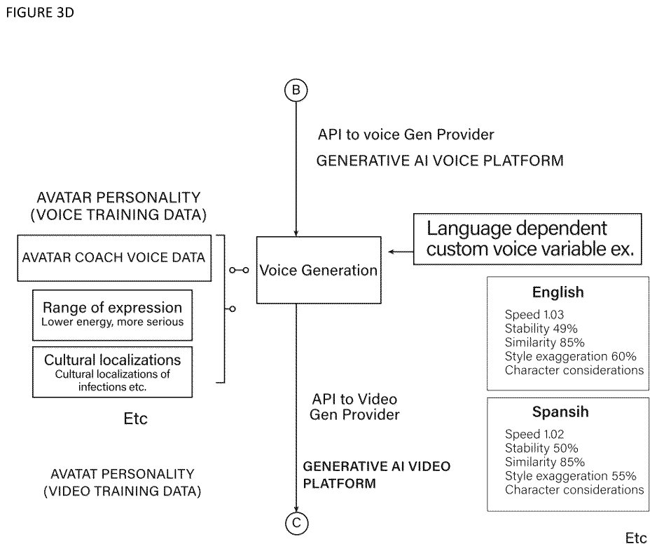

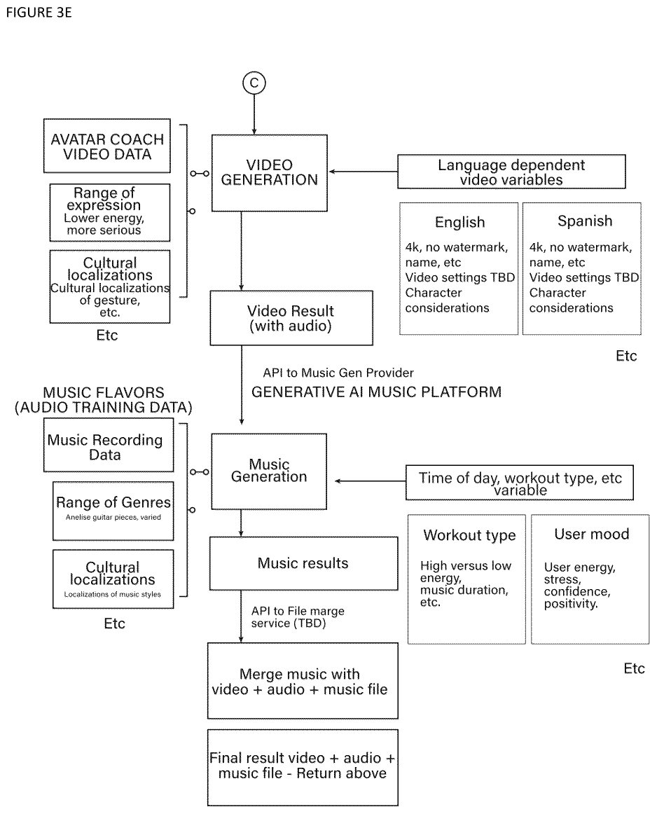

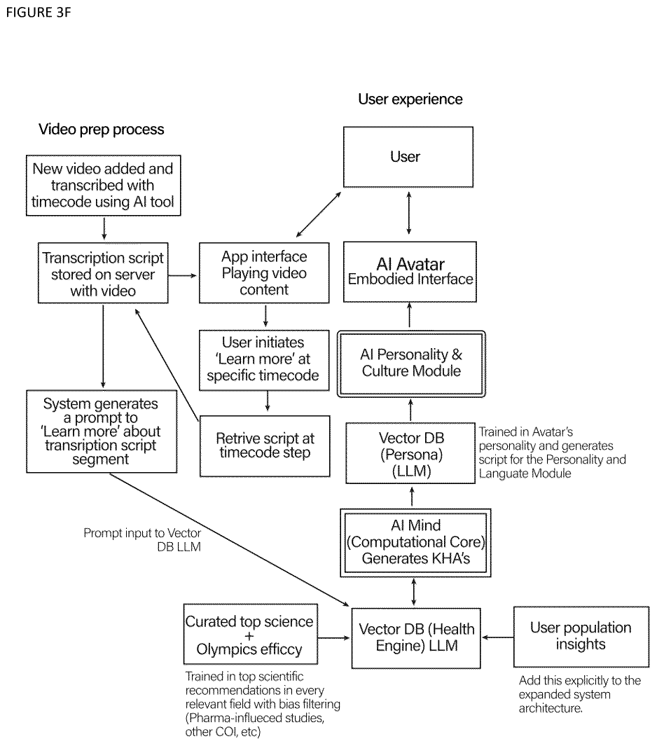

FIG. 3A-3F shows an exemplary avatar based coaching system. More details are in U.S. application Ser. No. 19/189,025, the content of which is incorporated by reference. A User (1) interacts with the system and receives personalized health coaching, ultimately leading to improved Health Outcomes (i). The user engages with the system through App Interfaces (2 and 5), which serve as digital touchpoints. The system collects Multi-Modal Health Data (3), including metrics such as sleep, nutrition, emotional state, glucose patterns, activity, and social dynamics. This data is processed by the AI Mind (Computational Core) (4), which acts as the central processing unit. The AI Mind synthesizes data and makes decisions using machine learning, large language models, and biometric modeling. The Vector DB (Persona) (LLM) (5) contains personality training data for the avatar, generating scripts for the AI Personality & Culture Module (6). This module processes personality aspects and cultural adaptations for the avatar's interactions. The AI Avatar Embodied Interface (7) serves as a programmatically generated, human-like guide that builds trust and emotional connection with the user. A Reinforcement Learning Model (ii) creates a closed feedback loop, allowing the system to evolve based on user responses and progress. The Vector DB (Health Engine) LLM (4b) is trained in top scientific recommendations across relevant health fields, further informing the AI Mind's decision-making process. This architecture enables the delivery of context-aware, behaviorally prioritized KHAs that can identify and correct underlying metabolic dysfunction. The system's ability to process and act on real-time inputs in ways that human coaches cannot match due to cognitive and logistical limitations makes it a unique and powerful tool for guiding human health and well-being. This system features a programmatically generated AI avatar that serves as the embodied interface between the user and a highly advanced health optimization engine, providing users with a trusted, human-like guide that delivers personalized coaching to improve their health, well-being, and longevity. Each avatar includes a customizable personality and appearance module, allowing for coaching personas-ranging from archetypal roles to branded or celebrity-style guides. This embodiment builds trust, emotional connection, and long-term engagement. Functionally, the avatar is powered by a dynamic computational “mind”: an AI system that delivers forward-prescribed KHAs based on continuously updated, multi-modal health data. These KHAs are synthesized using machine learning (ML), large language models (LLMs), and biometric modeling to process and act on real-time inputs such as sleep, nutrition, emotional state, glucose patterns, activity, and social dynamics. At its core, the system is designed to identify and correct underlying metabolic dysfunction—the root cause of many modern chronic conditions—by guiding behavior in precise, timely, and personalized ways. Unlike human coaches—who cannot cognitively or logistically integrate and respond to these data streams in real time—this system provides context-aware, behaviorally prioritized, and dynamically rescheduled actions, creating a closed feedback loop that evolves with the user. First validated in Olympic athletes, it contributed directly to improvements in metabolic function, glucose regulation, sleep quality, and performance readiness, leading to medal-winning outcomes. Far more than a digital assistant, this system constitutes a scalable, emotionally intelligent decision engine, uniquely positioned to guide human health in ways no human or conventional system can match.

The system supports sustainable fat loss rather than simple weight loss, with a primary focus on helping users achieve long-term fat reduction while preserving lean muscle mass and maintaining a high metabolic rate. This is especially important because many interventions, such as GLP-1 drugs, can inadvertently threaten muscle retention and metabolic health. Every dynamic aspect of the system—from meal calculations, timing, and digestion speed to the integration of strength training elements—is engineered to promote this sustainable fat loss outcome. The approach is never about calorie restriction; in fact, the system encourages users to avoid focusing on calories altogether, using calorie data only for backend calculations. Instead, users are guided to follow prescribed macronutrient targets at optimal times, particularly emphasizing proper breakfast composition and strategic fueling around workouts, as well as tailoring digestion speeds to individual needs. This methodology is designed to help users retain or build muscle, which in turn keeps metabolism elevated and supports long-term fat burning. The unique interface provides real-time feedback as users set their goals, reinforcing these principles. The system prioritizes slow-digesting, nutrient-dense meals with sufficient protein to ensure users feel satiated, avoid hunger battles, and preserve muscle mass. In contrast, traditional systems that focus on calorie restriction often reduce protein intake, leading to muscle loss, decreased metabolism, and ultimately making fat loss more difficult.

The system enables users to obtain their resting heart rate (RHR) directly within the app by tapping a button, even if they do not have external sensors or if sensor data is not available in their device's health kit. This is achieved using the phone's camera and a guided on-screen process, where the user places their finger over the camera and receives a real-time RHR measurement, leveraging validated smartphone-based photoplethysmography techniques. Once the RHR is captured, the system incorporates this data into the body composition goal calculator, using established associations between RHR, cardiorespiratory fitness, and body fat percentage. The RHR value helps inform the user's baseline fitness level and is factored into predictive models that estimate the time required to achieve specific body composition goals, such as muscle gain or fat loss. By integrating RHR data, the system personalizes recommendations and goal timelines, making them more accurate and actionable. This approach democratizes fitness tracking, allowing users without wearables to benefit from advanced health insights and progress monitoring. The RHR measurement is also used to dynamically adjust other aspects of the system, such as recommended workout intensity, recovery needs, and nutrition planning, ensuring that each user's plan is tailored to their current physiological state and long-term objectives.

This system features a programmatically generated AI avatar that acts as the primary interface between the user and a sophisticated health optimization engine. The user interacts with the system through various app interfaces, which serve as the main digital touchpoints for engaging with the AI coach and receiving personalized guidance. The AI avatar is not only visually embodied but also includes a customizable personality and appearance module, allowing users to select from a range of coaching personas, such as archetypal roles or branded and celebrity-style guides. This embodiment is designed to foster trust, emotional connection, and long-term engagement, making the avatar a relatable and motivating presence in the user's health journey. Functionally, the avatar is powered by a dynamic computational “mind”—an AI system that synthesizes multi-modal health data, including inputs related to sleep, nutrition, emotional state, glucose patterns, physical activity, and social dynamics. This data is continuously updated and processed by the AI Mind (Computational Core), which draws on a health engine vector database trained in leading scientific recommendations, as well as a persona vector database that informs the avatar's personality and communication style. The AI Personality & Culture Module further adapts the avatar's language and approach to fit the user's preferences and cultural context. The system's core function is to deliver forward-prescribed KHAs that are personalized, timely, and context-aware. These actions are generated using advanced machine learning, large language models, and biometric modeling, enabling the system to identify and address underlying metabolic dysfunction—the root cause of many chronic health conditions. Unlike human coaches, who cannot integrate and respond to these complex data streams in real time, this AI-driven system provides behaviorally prioritized and dynamically rescheduled actions, creating a closed feedback loop that evolves with the user through reinforcement learning.

Validated initially in Olympic athletes, the system has demonstrated measurable improvements in metabolic function, glucose regulation, sleep quality, and performance readiness, contributing to medal-winning outcomes. Ultimately, this system goes far beyond a digital assistant; it is a scalable, emotionally intelligent decision engine uniquely positioned to guide human health, well-being, and longevity in ways that no human or conventional system can match.

The system is an advanced, AI-powered health coaching platform designed to deliver personalized, emotionally intelligent guidance to users through a dynamic, embodied interface. At its core, the system begins with the user, who interacts via app interfaces that serve as the primary digital touchpoints. The user's multi-modal health data—including metrics such as sleep, nutrition, emotional state, glucose patterns, activity, and social dynamics—is continuously collected and fed into the AI Mind, the computational core of the system. This core processes the data using sophisticated machine learning models and is further informed by a health engine vector database trained on leading scientific recommendations.

For a deeply personalized experience, the AI Mind also accesses a separate vector database dedicated to the avatar's personality, which generates scripts tailored to the user's needs. These scripts are processed by the AI Personality & Culture Module, ensuring that the avatar's communication style and cultural context are finely tuned. The AI Avatar, serving as the system's embodied interface, delivers this tailored coaching directly to the user, fostering trust, emotional connection, and long-term engagement. The entire process is governed by a reinforcement learning model that creates a closed feedback loop, allowing the system to adapt and evolve based on user responses and health outcomes. This architecture enables the delivery of forward-prescribed, context-aware KHAs that are prioritized and dynamically rescheduled in response to real-time user data. Validated in high-performance contexts such as Olympic athletes, the system has demonstrated the ability to improve metabolic function, glucose regulation, sleep quality, and performance readiness. Ultimately, the system offers a scalable, emotionally intelligent decision engine that guides users toward optimal health, well-being, and longevity in ways unmatched by human coaches or traditional digital assistants.

1. A computer-implemented method for providing a computer avatar to deliver a personalized metabolic health plan, comprising:
receiving input data comprising user age, sex, body mass, goals, workout intensity, non-exercise activity thermogenesis, sleep timing and quality, stress response, and dietary patterns;
estimating resting metabolic rate using a weighted combination of metabolic rate estimation based on user physical parameters and organ system weight-based models;
determining a 24-hour calorie utilization profile by mapping metabolic rate slope and correlating feeding and activity events;
quantifying nutrient and metabolic gaps, including caloric deficit, macronutrient flow, hydration, and nourishment bottlenecks;
adjusting recommended targets for longevity, health, weight (fat) loss, lean tissue gain, and fitness by applying goal-specific tunable coefficients;
calculating a probability-ranked list of lifestyle interventions using additive, multiplicative, and weighted coefficient-based mathematical operations;
generating a visually accessible metabolic plan including at least one of: three to six key daily actions, pie charts of food or activity groups, and detailed units in grams, sets, or time intervals; and
providing an adaptive avatar interface for the avatar to engage a user in a conversational format, simplifies outputs, monitors adherence, and recalibrates recommendations in real time based on updated user data.

2. The method of claim 1, wherein estimating resting metabolic rate further comprises selecting equations dynamically based on body mass index and activity levels.

3. The method of claim 1, wherein feeding and activity events are represented by charted arrows on a timeline, each arrow proportionate to caloric intake or expenditure at the corresponding event.

4. The method of claim 1, wherein nutrient bottlenecks are identified for protein flow timing, carbohydrate distribution, omega-3, omega-6, omega-9 ratios, and vegetable intake, with user-specific recommendations generated for each.

5. The method of claim 1, further comprising dividing daily recommended macronutrient intake by typical meal frequency to estimate per-hour or per-meal nutritional targets.

6. The method of claim 1, wherein probability-ranked list calculation comprises scoring metabolic nutrition by combining additive components for positive actions and multiplicative penalties for bottlenecks.

7. The method of claim 1, wherein real-time recalibration of recommendations occurs when deviation in sleep timing or quality is detected, modifying recovery window and intervention priority accordingly.

8. The method of claim 1, wherein the adaptive avatar interface is selected from the group consisting of three-dimensional graphical avatars, voice-based agents, and text-based agents.

9. The method of claim 1, wherein the metabolic plan includes individualized recommendations for movement and exercise, including timing, intensity, and duration, adjusted according to user goals.

10. The method of claim 1, wherein the metabolic rate estimation equation based on user physical parameters validated metabolic rate estimation uses the Mifflin-St. Jeor equation.

11. The method of claim 1, further comprising identifying and ranking the top three nutrition, movement, or sleep adjustments required to achieve selected user goals.

12. The method of claim 1, wherein gap charting includes graphical representation of both caloric and specific nutrient deficits over a 24-hour slope.

13. The method of claim 1, wherein recommendations for food portions are provided using standard hand metrics including thumb, palm, and first measures.

14. The method of claim 1, wherein recommendations for exercise refueling are tuned to user body weight and post-exercise metabolic requirements, with timing windows for optimal nutrient absorption.

16. The method of claim 1, wherein a plurality of alternative lifestyle scaffolds are selectable by the user, including default Mediterranean diet modification and extreme carbohydrate variation options.

17. The method of claim 1, comprising determining a user target estimate using:

| | Weight/fat | | | | Healthy Wt |
|---|---|---|---|---|---|
| Goal: | loss WL | Longevity L | Health H | Fitness F | Gain WG |
| Z = importance | Z_{WL}= | Z_{L}= | Z_{H}= | Z_{F}= | Z_{WG}= |
| for change 0-10 | | | | | |
| Potential loss of | Muscle, | Muscle, | Fast fat | Health | Leanness |
| | health | fitness | loss/fitness | | |
| Shift daily Cal up, | DOWN by | DOWN by | 10 × Z_{H} = % of | UP to | UP by |
| down, or | 2.5 × Z_{WL} %= | 1 × Z_{L} %= | processed Cal | recover | (1-2.5) × Z_{WG} %= |
| averaged by a % | | | that must shift | 1 × Z_{F} %= | |
| of daily | | | to quality food | | |
| metabolic rate | | | | | |

18. The method of claim 1, comprising determining user intensity levels of low (L), medium (M), high (H) and very high (V) by how long the user maintains exercise before exhaustion without injury risk and maintaining normal movement patterns, wherein the user maintains the very high intensity for up to 30 min maximum, high 60 min, medium 2 hours, and low up to 4 hours maximum before exhaustion.

19. A method to render an avatar, comprising:
receiving user preferences and exercise data with a large language model (LLM), the LLM generating content to be rendered to a user; and
rendering the avatar with a photo-realistic video renderer to coach the user on nutrition, wherein the LLM determines a personalized metabolic plan for a user by:
receiving user data, the data comprising age, sex, body weight, body height, activity level, body mass index, metabolic goals, sleep timing, sleep quality, stress patterns, and dietary intake patterns;
estimating a resting metabolic rate for the user by selectively applying one or more validated metabolic equations, each equation configurable to utilize user-specific physical inputs and optionally forming a weighted average of outputs; wherein the equations comprise at least weight, height, age, and sex variables;
calculating an estimated total daily metabolic rate by incorporating the resting metabolic rate and adjusting for user-specific activity levels, non-exercise activity thermogenesis, and weighted goal preferences;
determining calorie utilization for a defined 24-hour period, by dividing the total daily metabolic rate by 24 to produce an hourly metabolic rate, and mapping user feeding events and activity events on a timeline, wherein feeding events are assigned upward graphical indicators sized proportional to caloric intake, and activity events are assigned downward graphical indicators sized proportional to caloric expenditure;
quantifying caloric gaps by comparing daily caloric intake at feeding and snacking events with total expenditure, such that cumulative deficits or surpluses are represented graphically over the 24-hour period from postprandial sleep state through daily activity cycles;
repeating the gap quantification and mapping process for individual macronutrients including protein and carbohydrate, by calculating user baseline needs for each macronutrient, computing a healing or recovery adjustment as a function of recent injury, physical stress or exercise, and determining timing and distribution across meals, snacks, and feeding events;
estimating meal and snack portion sizes for caloric and macronutrient targets using visual and physical measures including thumb, palm, and first volumes, and aligning portion guidelines with user preferences and patterns;
assigning tunable goal values for each metabolic objective, the objective comprising at least weight and fat loss, longevity, health, fitness, healthy weight gain, and lean tissue protection, each goal associated with a user-adjustable Z-value indicative of relative importance; wherein Z-values are used quantitatively to increase or decrease recommended caloric and nutrient targets by fixed or proportionate amounts;
calculating nutrient bottlenecks and gaps including protein, carbohydrate, hydration, vegetable intake, and omega fatty acid intake; for each bottleneck, determining a recommended intake range and identifying food sources and portion sizes to address the gap;
estimating hydration requirements in relation to metabolic rate, recommending a baseline target of liquid intake per 1000 calories of estimated metabolic rate, and generating timing recommendations for intake distribution throughout the day;
mapping timing for slow-digesting and fast-digesting foods based on digestion rates for protein, carbohydrate, and fat sources, and determining minimum and ideal hourly nutritional requirements by dividing daily targets by estimated effective provision periods between feedings;
calculating and visualizing vegetable and fatty acid goals, including baseline and Z-value-driven increases, and presenting results as portion guidelines (in cups, palms, tablespoons);
recommending and displaying individualized metabolic meal plans for the user, including selection and arrangement of food libraries for each meal and snack event, with checklists and graphical meal composition options;
scoring user nutritional and metabolic adherence using carrot versus stick scoring algorithms, wherein positive actions are counted additively and major bottlenecks result in multiplicative penalties, producing a composite metabolic health score;
recommending movement and exercise interventions, including activity break scheduling, specific exercise and activity recommendations are modulated by user-selected Z-values corresponding to their metabolic goals;
recommending sleep, stress, eating, hydration, movement, and psychological patterns for the user, prompting user enrollment of individual priority factors for each category, ranking factors by combined importance and difficulty, and outlining top-priority actions with recommended solutions for common barriers;
generating an adaptive, visually accessible output comprising at least: (i) daily key accomplishment checklists; (ii) graphical pie charts illustrating proportional metabolic targets; and (iii) detailed units of food portions or activity sets in grams, ounces, thumb/palm/fist equivalents, minutes, or repetitions; and
providing real-time tracking and adaptive recalibration of recommendations and targets, updating outputs and priority interventions as user data, adherence, or underlying conditions (including sleep quality and activity deviation) change.
